(19) United States
(12) Patent Application Publication
(10) Pub. No.: US 2026/0325139 A1
(43) Pub. Date: Oct. 22, 2026

(54) OCCLUSION SHEATHS CONFIGURED FOR PERCUTANEOUS VASCULAR ACCESS

(71) Applicant: Silk Road Medical, Inc., Sunnyvale, CA (US)

(72) Inventors: Brad Steele, Sunnyvale, CA (US); Abigail Brooks, Sunnyvale, CA (US); Blaine Schneider, Sunnyvale, CA (US)

(73) Assignee: Silk Road Medical, Inc., Sunnyvale, CA (US)

(21) Appl. No.: 18/997,144

(22) PCT Filed: Jul. 20, 2023
(86) PCT No.: PCT/US2023/028248

§ 371 Date: Jan. 20, 2025

Related U.S. Application Data
(60) Provisional application No. 63/391,067, filed on Jul. 21, 2022.

(51) Int. Cl. A61B 17/12; A61M 1/36; A61M 25/00; A61M 25/10

(52) U.S. Cl. CPC A61B 17/12136; A61B 17/12109; A61M 1/3666; A61B 2017/1205; A61M 2025/0006; A61M 2025/1095

(57) ABSTRACT

Systems and method are configured for vascular and/or neurointerventional procedures. The systems and methods enable safe and rapid access, including percutaneous access, to the carotid artery and further to the cerebral or intracranial arteries for the introduction of interventional devices such as to treat disease conditions. The methods and devices can include a vascular access and retrograde flow system.

### CROSS-REFERENCE TO RELATED APPLICATION

The current application claims priority under 35 U.S.C. § 119(e) to U.S. Provisional patent application Ser. No. 63/391,067, filed on Jul. 21, 2022, and entitled “OCCLUSION SHEATHS CONFIGURED FOR PERCUTANEOUS VASCULAR ACCESS,” which is incorporated by reference herein in its entirety.

### BACKGROUND

The present disclosure relates generally to medical methods and devices.

More particularly, the present disclosure relates to methods, system, and devices for accessing and treating carotid arterial vasculature and optionally establishing retrograde blood flow during performance of carotid artery stenting and other procedures.

The present disclosure also relates to methods and systems for accessing and treating the cerebral arterial vasculature such as for the treatment of stroke, Intracranial Atherosclerotic Disease (ICAD), transient ischemic attack (TIA), acute ischemic stroke (AIS), tandem lesions, ruptured and unruptured intra-and extra-cranial aneurysm embolization, chronic occlusions, and other disease conditions of the neurovasculature.

### SUMMARY

Disclosed are methods and devices for vascular and/or neurointerventional procedures. The methods and devices enable safe and rapid access, including percutaneous access, to the carotid artery and further to the cerebral or intracranial arteries for the introduction of interventional devices such as to treat stroke and/or other disease conditions. The methods and devices include a vascular access and retrograde flow system.

In one aspect, there is disclosed a system for use in accessing and treating a carotid artery, the system comprising: an outer guide sheath configured to be percutaneously delivered into a carotid artery; an inner catheter movably positioned inside the outer guide sheath, the inner catheter having an expandable element positioned on a distal region of the inner catheter, wherein the inner catheter comprises a lumen extending between a proximal end and a distal end adapted to receive blood flow from a common carotid artery, the outer guide sheath and inner catheter adapted to be collectively introduced into the common carotid artery, the expandable element adapted to expand and occlude the common carotid artery; and a locking collar positioned on a proximal region of the outer guide sheath, the locking collar configured to rotate about an outer wall of the outer guide sheath, wherein the locking collar has a first set of threads on an inner wall of the locking collar, and wherein the first set of threads engage a second set of threads on an outer wall of the inner catheter, and wherein the locking collar rotates to cause the first set of threads to engage the second set of threads and lock a position of the inner catheter relative to the outer guide sheath; wherein the inner catheter is movably positioned inside the outer guide sheath between a first position wherein the outer guide sheath is positioned relative to the outer guide sheath such that a portion of the outer guide sheath covers and constrains the expandable element in a contracted state, and a second position wherein outer guide sheath does not constrains the expandable element, and wherein the locking collar aligns with the second set of threads when the inner catheter is in the first position

Other aspects, features and advantages should be apparent from the following description of various embodiments, which illustrate, by way of example, the principles of the disclosure.

### BRIEF DESCRIPTION OF THE DRAWINGS

FIG. 1A is a schematic illustration of a retrograde blood flow system including a flow control assembly wherein an arterial access device accesses the common carotid artery via a transcarotid approach and a venous return device communicates with the internal jugular vein.

FIG. 1B is a schematic illustration of a retrograde blood flow system wherein an arterial access device accesses the common carotid artery via a transcarotid approach and a venous return device communicates with the femoral vein.

FIG. 1C is a schematic illustration of a retrograde blood flow system wherein an arterial access device accesses the common carotid artery via a transfemoral approach and a venous return device communicates with the femoral vein.

FIG. 1D is a schematic illustration of a retrograde blood flow system wherein retrograde flow is collected in an external receptacle.

FIG. 2A is an enlarged view of the carotid artery wherein the carotid artery is occluded with an occlusion element on the sheath and connected to a reverse flow shunt, and an interventional device, such as a stent delivery system or other working catheter, is introduced into the carotid artery via an arterial access device.

FIG. 2B is an alternate system wherein the carotid artery is occluded with a separate external occlusion device and connected to a reverse flow shunt, and an interventional device, such as a stent delivery system or other working catheter, is introduced into the carotid artery via an arterial access device.

FIG. 3 is an alternate system wherein the carotid artery is connected to a reverse flow shunt and an interventional device, such as a stent delivery system or other working catheter, is introduced into the carotid artery via an arterial access device, and the carotid artery is occluded with a separate occlusion device.

FIG. 4 illustrates a normal cerebral circulation diagram including the Circle of Willis

FIG. 5 illustrates the vasculature in a patient's neck, including the common carotid artery CCA, the internal carotid artery ICA, the external carotid artery ECA, and the internal jugular vein IJV.

FIG. 6A illustrates an arterial access device useful in the methods and systems of the present disclosure.

FIG. 6B illustrates an additional arterial access device construction with a reduced diameter distal end.

FIGS. 7A and 7B illustrate a tube useful with the sheath of FIG. 6A.

FIG. 7C show an embodiment of a sheath stopper.

FIG. 7D shows the sheath stopper of FIG. 7C positioned on a sheath.

FIG. 8A illustrates an additional arterial access device construction with an expandable occlusion element.

FIG. 8B illustrates an additional arterial access device construction with an expandable occlusion element and a reduced diameter distal end.

FIGS. 9A and 9B illustrate an additional embodiment of an arterial access device.

FIGS. 9C and 9D illustrate an embodiment of a valve on the arterial access device.

FIGS. 10A through 10C and FIG. 11 illustrate embodiments of a venous return device useful in the methods and systems of the present disclosure.

FIG. 12 illustrates the system of FIG. 1 including a flow control assembly.

FIGS. 13-14 illustrate an embodiment of a variable flow resistance component useful in the methods and systems of the present disclosure.

FIGS. 15A-15C show schematic representations of an embodiment of a sheath 1505 that is configured for percutaneous access and occlusion of a blood vessel such as an artery.

FIGS. 16A and 16B show schematic representations of a distal region of an embodiment of a sheath that is configured for percutaneous access and occlusion of a blood vessel.

FIGS. 17A and 17B show schematic representations of a distal region of an embodiment of a sheath 1705 that is configured for percutaneous access and occlusion of a blood vessel.

FIG. 18A show an embodiment of a sheath having a distal umbrella element.

FIG. 18B shows a cross-sectional view of the sheath of FIG. 18A.

FIGS. 19A and 19B show another embodiment of an umbrella element on a sheath.

FIGS. 20A and 20B show a distal region of a sheath having an expandable element such as a balloon.

FIGS. 21A and 21B show another embodiment of a sheath that includes an expandable balloon.

FIGS. 22A-22C show an embodiment of a sheath that includes an expandable balloon integrated into an outer wall of the sheath.

FIGS. 23A-23B show a proximal hub of a sheath configured to controlling tension configuration of a balloon.

FIGS. 24A and 24B show an alternate mechanism that can be coupled to the sheath for controlling the tension configuration of the balloon.

FIGS. 25A and 25B show an embodiment of a sheath system that includes a balloon sheath integrated with a guide sheath.

FIGS. 26A-26C show another embodiment of a sheath system that includes a balloon sheath integrated with a guide sheath.

FIGS. 27 and 28 shows schematic, cross-sectional views of an embodiments of the sheath.

FIGS. 29A-30B show embodiments of a sheath positioned in a blood vessel.

### DETAILED DESCRIPTION

The disclosed methods, apparatus, and systems establish and facilitate retrograde or reverse flow blood circulation in the region of the carotid artery bifurcation in order to limit or prevent the release of emboli into the cerebral vasculature, particularly into the internal carotid artery. In non-limiting examples, the methods are useful for interventional procedures, such as stenting and angioplasty, atherectomy, performed through a transcarotid approach or transfemoral into the common carotid artery, either using an open surgical technique or using a percutaneous technique, such as a modified Seldinger technique or a micropuncture technique. Any of a wide variety of interventions can be performed in conjunction with the systems and method described herein including the treatment of stroke, Intracranial Atherosclerotic Disease (ICAD), transient ischemic attack (TIA), acute ischemic stroke (AIS), tandem lesions, ruptured and unruptured intra-and extra-cranial aneurysm embolization, chronic occlusions, Intravascular Lithotripsy (IVL), Shockwave Intravascular Lithotripsy (IVL), and other disease conditions of the neurovasculature

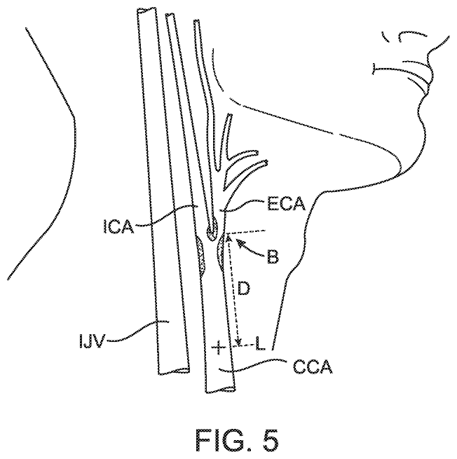

Access into the common carotid artery (such as shown in FIG. 5) is established by placing an access sheath or other tubular access cannula into a lumen of the artery, typically having a distal end of the sheath positioned proximal to the junction or bifurcation B from the common carotid artery to the internal and external carotid arteries. A percutaneous version of the sheath may have an occlusion member at the distal end, for example a compliant occlusion balloon. A catheter or guidewire with an occlusion member, such as a balloon, may be placed through the access sheath and positioned in the proximal external carotid artery ECA to inhibit the entry of emboli, but occlusion of the external carotid artery is usually not necessary. A second return sheath is placed in the venous system, for example the internal jugular vein IJV or femoral vein FV. The arterial access and venous return sheaths are connected to create an external arterial-venous shunt.

Retrograde flow is established and modulated to meet the patient's requirements. Flow through the common carotid artery is occluded, either with an external vessel loop or tape, a vascular clamp, an internal occlusion member such as a balloon, or other type of occlusion means. When flow through the common carotid artery is blocked, the natural pressure gradient between the internal carotid artery and the venous system will cause blood to flow in a retrograde or reverse direction from the cerebral vasculature through the internal carotid artery and through the shunt into the venous system.

Alternately, the venous sheath can be eliminated and the arterial sheath could be connected to an external collection reservoir or receptacle. The reverse flow could be collected in this receptacle. If desired, the collected blood could be filtered and subsequently returned to the patient during or at the end of the procedure. The pressure of the receptacle could be open to atmospheric pressure, causing the pressure gradient to create blood to flow in a reverse direction from the cerebral vasculature to the receptacle or the pressure of the receptacle could be a negative pressure.

Optionally, to achieve or enhance reverse flow from the internal carotid artery, flow from the external carotid artery may be blocked, typically by deploying a balloon or other occlusion element in the external carotid just above (i.e., distal) the bifurcation within the internal carotid artery.

Although the procedures and protocols described hereinafter will be particularly directed at carotid stenting, it will be appreciated that the methods for accessing the carotid artery described herein would also be useful for angioplasty, artherectomy, and any other interventional procedures which might be carried out in the carotid arterial system, such as at a location near the bifurcation between the internal and external carotid arteries. In addition, it will be appreciated that some of these access, vascular closure, and embolic protection methods will be applicable in other vascular interventional procedures, for example the treatment of acute stroke.

The present disclosure includes a number of specific aspects for improving the performance of carotid artery access protocols. At least some of these individual aspects and improvements can be performed individually or in combination with one or more other of the improvements in order to facilitate and enhance the performance of the particular interventions in the carotid arterial system.

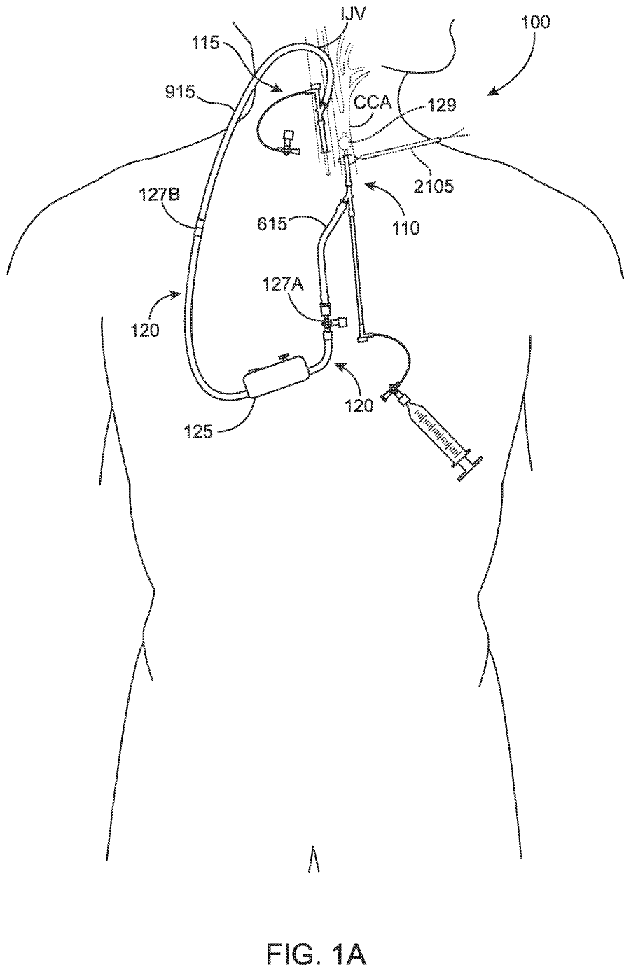

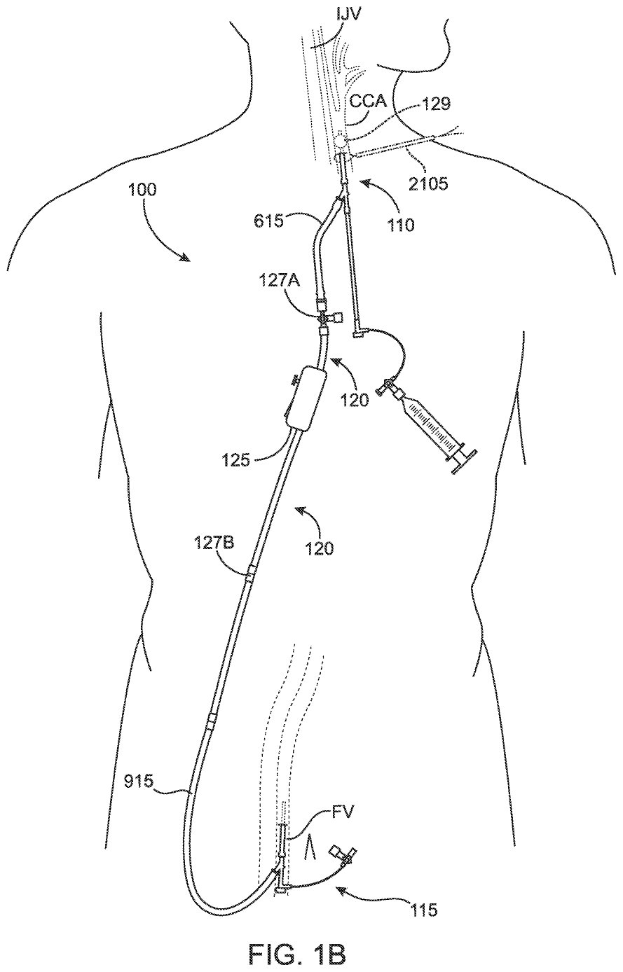

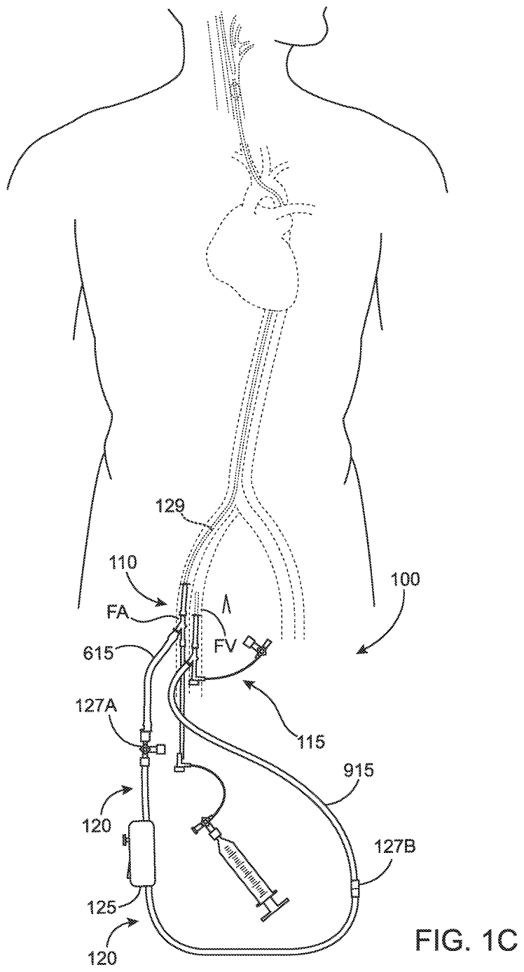

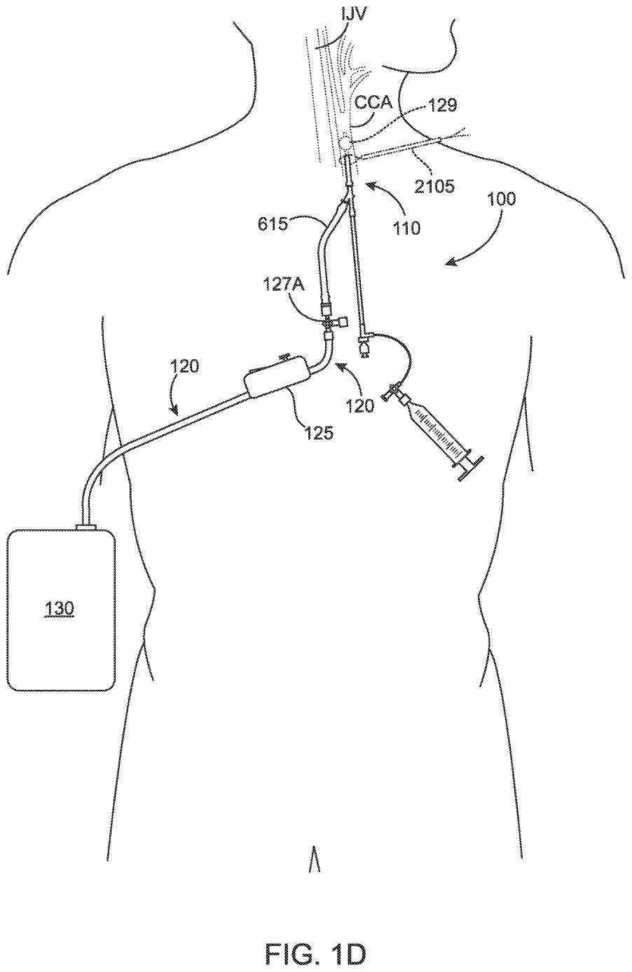

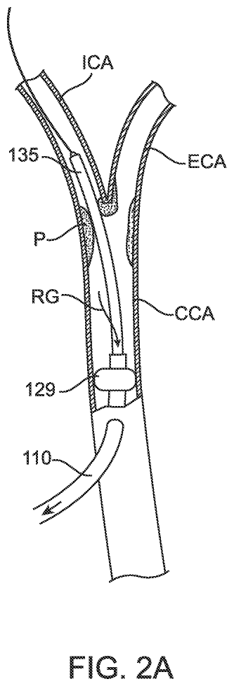

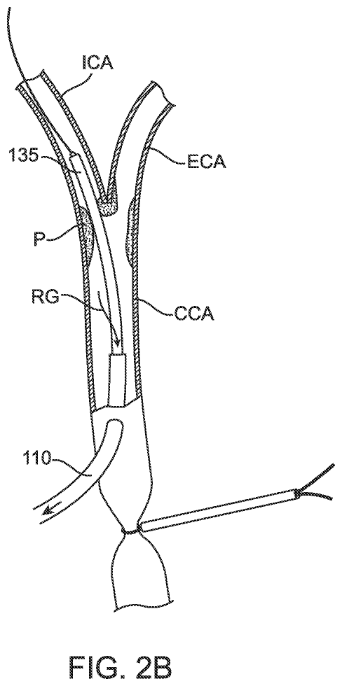

FIG. 1A shows a first embodiment of a retrograde flow system 100 that is adapted to establish and facilitate retrograde or reverse flow blood circulation in the region of the carotid artery bifurcation in order to limit or prevent the release of emboli into the cerebral vasculature, particularly into the internal carotid artery. The system 100 interacts with the carotid artery to provide retrograde flow from the carotid artery to a venous return site, such as the internal jugular vein (or to another return site such as another large vein or an external receptacle in alternate embodiments.) The retrograde flow system 100 includes an arterial access device 110, a venous return device 115, and a shunt 120 that provides a passageway for retrograde flow from the arterial access device 110 to the venous return device 115. A flow control assembly 125 interacts with the shunt 120. The flow control assembly 125 is adapted to regulate and/or monitor the retrograde flow from the common carotid artery to the internal jugular vein, as described in more detail below. The flow control assembly 125 interacts with the flow pathway through the shunt 120, either external to the flow path, inside the flow path, or both. The arterial access device 110 at least partially inserts into the common carotid artery CCA and the venous return device 115 at least partially inserts into a venous return site such as the internal jugular vein IJV, as described in more detail below. The arterial access device 110 and the venous return device 115 couple to the shunt 120 at connection locations 127a and 127b. When flow through the common carotid artery is blocked, the natural pressure gradient between the internal carotid artery and the venous system causes blood to flow in a retrograde or reverse direction RG (FIG. 2A) from the cerebral vasculature through the internal carotid artery and through the shunt 120 into the venous system. The flow control assembly 125 modulates, augments, assists, monitors, and/or otherwise regulates the retrograde blood flow.

In the embodiment of FIG. 1A, the arterial access device 110 accesses the common carotid artery CCA via a transcarotid approach. Transcarotid access provides a short length and non-tortuous pathway from the vascular access point to the target treatment site thereby easing the time and difficulty of the procedure, compared for example to a transfemoral approach. In an embodiment, the arterial distance from the arteriotomy to the target treatment site (as measured traveling through the artery) is 15 cm or less. In an embodiment, the distance is between 5 and 10 cm. Additionally, this access route reduces the risk of emboli generation from navigation of diseased, angulated, or tortuous aortic arch or common carotid artery anatomy. At least a portion of the venous return device 115 is placed in the internal jugular vein IJV. In an embodiment, transcarotid access to the common carotid artery is achieved percutaneously via an incision or puncture in the skin through which the arterial access device 110 is inserted. If an incision is used, then the incision can be about 0.5 cm in length. An occlusion element 129, such as an expandable balloon, can be used to occlude the common carotid artery CCA at a location proximal of the distal end of the arterial access device 110. The occlusion element 129 can be located on the arterial access device 110 or it can be located on a separate device. In an alternate embodiment, the arterial access device 110 accesses the common carotid artery CCA via a direct surgical transcarotid approach. In the surgical approach, the common carotid artery can be occluded using a tourniquet 2105. The tourniquet 2105 is shown in phantom to indicate that it is a device that is used in the optional surgical approach.

In another embodiment, shown in FIG. 1B, the arterial access device 110 accesses the common carotid artery CCA via a transcarotid approach while the venous return device 115 access a venous return site other than the jugular vein, such as a venous return site comprised of the femoral vein FV. The venous return device 115 can be inserted into a central vein such as the femoral vein FV via a percutaneous puncture in the groin.

In another embodiment, shown in FIG. 1C, the arterial access device 110 accesses the common carotid artery via a femoral approach. According to the femoral approach, the arterial access device 110 approaches the CCA via a percutaneous puncture into the femoral artery FA, such as in the groin, and up the aortic arch AA into the target common carotid artery CCA. The venous return device 115 can communicate with the jugular vein JV or the femoral vein FV.

FIG. 1D shows yet another embodiment, wherein the system provides retrograde flow from the carotid artery to an external receptacle 130 rather than to a venous return site. The arterial access device 110 connects to the receptacle 130 via the shunt 120, which communicates with the flow control assembly 125. The retrograde flow of blood is collected in the receptacle 130. If desired, the blood could be filtered and subsequently returned to the patient. The pressure of the receptacle 130 could be set at zero pressure (atmospheric pressure) or even lower, causing the blood to flow in a reverse direction from the cerebral vasculature to the receptacle 130. Optionally, to achieve or enhance reverse flow from the internal carotid artery, flow from the external carotid artery can be blocked, typically by deploying a balloon or other occlusion element in the external carotid artery just above the bifurcation with the internal carotid artery. FIG. 1D shows the arterial access device 110 arranged in a transcarotid approach with the CCA although it should be appreciated that the use of the external receptacle 130 can also be used with the arterial access device 110 in a transfemoral approach.

With reference to the enlarged view of the carotid artery in FIG. 2A, a therapeutic or interventional device, such as a stent delivery system 135 or other working catheter, can be introduced into the carotid artery via the arterial access device 110 or percutaneous sheath, as described in detail below. The stent delivery system 135 can be used to treat the plaque P such as to deploy a stent into the carotid artery. The arrow RG in FIG. 2A represents the direction of retrograde flow. As shown in FIG. 2B, in an alternate embodiment a clamp element is used to occlude the artery.

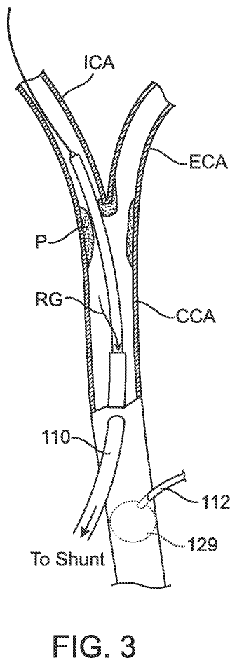

FIG. 3 shows an alternative embodiment, wherein the occlusion element 129 can be introduced into the carotid artery on a second sheath 112 separate from the distal sheath 605 of the arterial access device 110. The second or “proximal” sheath 112 can be adapted for insertion into the common carotid artery in a proximal or “downward” direction away from the cerebral vasculature. The second, proximal sheath can include an inflatable balloon 129 or other occlusion element, generally as described above. The distal sheath 605 of the arterial access device 110 can be then placed into the common carotid artery distal of the second, proximal sheath and generally oriented in a distal direction toward the cerebral vasculature. By using separate occlusion and access sheaths, the size of the arteriotomy needed for introducing the access sheath can be reduced.

### DESCRIPTION OF ANATOMY

### Collateral Brain Circulation

The Circle of Willis CW is the main arterial anastomatic trunk of the brain where all major arteries which supply the brain, namely the two internal carotid arteries (ICAs) and the vertebral basilar system, connect. The blood is carried from the Circle of Willis by the anterior, middle and posterior cerebral arteries to the brain. This communication between arteries makes collateral circulation through the brain possible.

Blood flow through alternate routes is made possible thereby providing a safety mechanism in case of blockage to one or more vessels providing blood to the brain. The brain can continue receiving adequate blood supply in most instances even when there is a blockage somewhere in the arterial system (e.g., when the ICA is ligated as described herein). Flow through the Circle of Willis ensures adequate cerebral blood flow by numerous pathways that redistribute blood to the deprived side.

The collateral potential of the Circle of Willis is believed to be dependent on the presence and size of its component vessels. It should be appreciated that considerable anatomic variation between individuals can exist in these vessels and that many of the involved vessels may be diseased. For example, some people lack one of the communicating arteries. If a blockage develops in such people, collateral circulation is compromised resulting in an ischemic event and potentially brain damage. In addition, an autoregulatory response to decreased perfusion pressure can include enlargement of the collateral arteries, such as the communicating arteries, in the Circle of Willis. An adjustment time is occasionally required for this compensation mechanism before collateral circulation can reach a level that supports normal function. This autoregulatory response can occur over the space of 15 to 30 seconds and can only compensate within a certain range of pressure and flow drop. Thus, it is possible for a transient ischemic attack to occur during the adjustment period. Very high retrograde flow rate for an extended period of time can lead to conditions where the patient's brain is not getting enough blood flow, leading to patient intolerance as exhibited by neurologic symptoms or in some cases a transient ischemic attack.

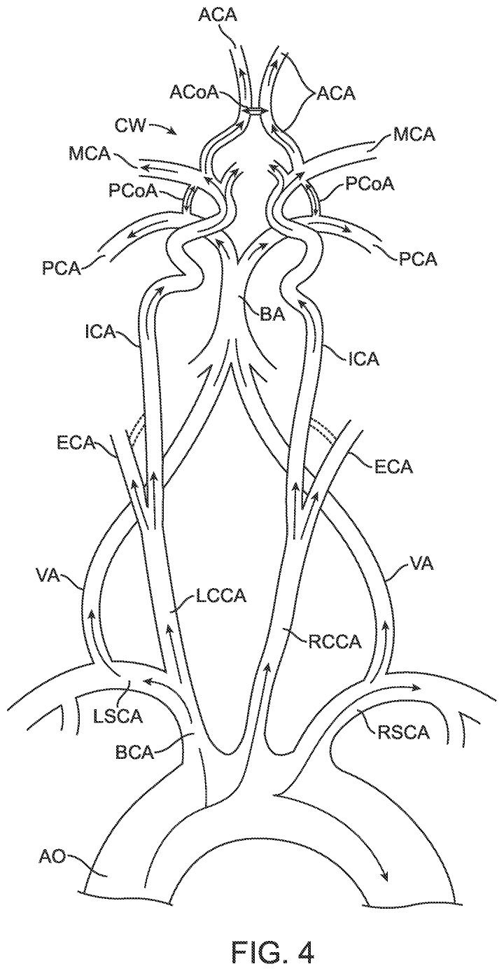

FIG. 4 depicts a normal cerebral circulation and formation of Circle of Willis CW. The aorta AO gives rise to the brachiocephalic artery BCA, which branches into the left common carotid artery LCCA and left subclavian artery LSCA. The aorta AO further gives rise to the right common carotid artery RCCA and right subclavian artery RSCA. The left and right common carotid arteries CCA gives rise to internal carotid arteries ICA which branch into the middle cerebral arteries MCA, posterior communicating artery PcoA, and anterior cerebral artery ACA. The anterior cerebral arteries ACA deliver blood to some parts of the frontal lobe and the corpus striatum. The middle cerebral arteries MCA are large arteries that have tree-like branches that bring blood to the entire lateral aspect of each hemisphere of the brain. The left and right posterior cerebral arteries PCA arise from the basilar artery BA and deliver blood to the posterior portion of the brain (the occipital lobe).

Anteriorly, the Circle of Willis is formed by the anterior cerebral arteries ACA and the anterior communicating artery ACoA which connects the two ACAs. The two posterior communicating arteries PCoA connect the Circle of Willis to the two posterior cerebral arteries PCA, which branch from the basilar artery BA and complete the Circle posteriorly.

The common carotid artery CCA also gives rise to external carotid artery ECA, which branches extensively to supply most of the structures of the head except the brain and the contents of the orbit. The ECA also helps supply structures in the neck and face.

### Carotid Artery Bifurcation

FIG. 5 shows an enlarged view of the relevant vasculature in the patient's neck. The common carotid artery CCA branches at bifurcation B into the internal carotid artery ICA and the external carotid artery ECA. The bifurcation is located at approximately the level of the fourth cervical vertebra. FIG. 5 shows plaque P formed at the bifurcation B.

As discussed above, the arterial access device 110 can access the common carotid artery CCA via a transcarotid approach. Pursuant to the transcarotid approach, the arterial access device 110 is inserted into the common carotid artery CCA at an arterial access location L, which can be, for example, a surgical incision or puncture in the wall of the common carotid artery CCA. There is typically a distance D of around 5 to 7 cm between the arterial access location L and the bifurcation B. When the arterial access device 110 is inserted into the common carotid artery CCA, it is undesirable for the distal tip of the arterial access device 110 to contact the bifurcation B as this could disrupt the plaque P and cause generation of embolic particles. In order to minimize the likelihood of the arterial access device 110 contacting the bifurcation B, in an embodiment only about 2-4 cm of the distal region of the arterial access device is inserted into the common carotid artery CCA during a procedure.

The common carotid arteries are encased on each side in a layer of fascia called the carotid sheath. This sheath also envelops the internal jugular vein and the vagus nerve. Anterior to the sheath is the sternocleidomastoideole. Transcarotid access to the common carotid artery and internal jugular vein, either percutaneous or surgical, can be made immediately superior to the clavicle, between the two heads of the sternocleidomastoid muscle and through the carotid sheath, with care taken to avoid the vagus nerve.

At the upper end of this sheath, the common carotid artery bifurcates into the internal and external carotid arteries. The internal carotid artery continues upward without branching until it enters the skull to supply blood to the retina and brain. The external carotid artery branches to supply blood to the scalp, facial, ocular, and other superficial structures.

Intertwined both anterior and posterior to the arteries are several facial and cranial nerves.

Additional neck muscles may also overlay the bifurcation. These nerve and muscle structures can be dissected and pushed aside to access the carotid bifurcation during a carotid endarterectomy procedure. In some cases the carotid bifurcation is closer to the level of the mandible, where access is more challenging and with less room available to separate it from the various nerves which should be spared. In these instances, the risk of inadvertent nerve injury can increase and an open endarterectomy procedure may not be a good option.

### RETROGRADE BLOOD FLOW SYSTEM

As discussed, the retrograde flow system 100 includes the arterial access device 110, venous return device 115, and shunt 120 which provides a passageway for retrograde flow from the arterial access device 110 to the venous return device 115. The system also includes the flow control assembly 125, which interacts with the shunt 120 to regulate and/or monitor retrograde blood flow through the shunt 120. Exemplary embodiments of the components of the retrograde flow system 100 are now described.

### Arterial Access Device

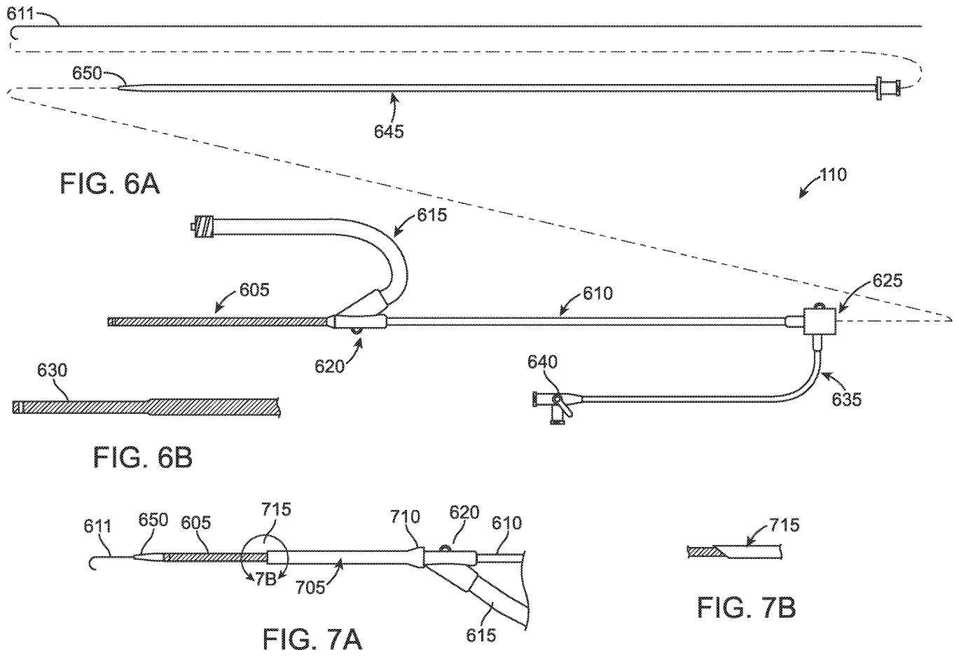

FIG. 6A shows an exemplary embodiment of the arterial access device 110, which comprises a distal sheath 605, a proximal extension 610, a flow line 615, an adaptor or Y-connector 620, and a hemostasis valve 625. The arterial access device may also comprise a dilator 645 with a tapered tip 650 and an introducer guide wire 611. The arterial access device together with the dilator and introducer guidewire are used together to gain access to a vessel. Features of the arterial access device may be optimized for transcarotid access. For example, the design of the access device components may be optimized to limit the potential injury on the vessel due to a sharp angle of insertion, allow atraumatic and secure sheath insertion, and limiting the length of sheath, sheath dilator, and introducer guide wire inserted into the vessel. The arterial access device 110 can include or comprise any embodiment of the percutaneous sheaths described herein.

The distal sheath 605 is adapted to be introduced through an incision or puncture in a wall of a common carotid artery, either an open surgical incision or a percutaneous puncture established, for example, using the Seldinger technique. The length of the sheath can be in the range from 5 to 15 cm, usually being from 10 cm to 12 cm. The inner diameter is typically in the range from 7 Fr (1 Fr=0.33 mm), to 10 Fr, usually being 8 Fr. Particularly when the sheath is being introduced through the transcarotid approach, above the clavicle but below the carotid bifurcation, it is desirable that the sheath 605 be highly flexible while retaining hoop strength to resist kinking and buckling. Thus, the distal sheath 605 can be circumferentially reinforced, such as by braid, helical ribbon, helical wire, cut tubing, or the like and have an inner liner so that the reinforcement structure is sandwiched between an outer jacket layer and the inner liner. The inner liner may be a low friction material such as PTFE. The outer jacket may be one or more of a group of materials including Pebax, thermoplastic polyurethane, or nylon. In an embodiment, the reinforcement structure or material and/or outer jacket material or thickness may change over the length of the sheath 605 to vary the flexibility along the length. In an alternate embodiment, the distal sheath is adapted to be introduced through a percutaneous puncture into the femoral artery, such as in the groin, and up the aortic arch AA into the target common carotid artery CCA.

The distal sheath 605 can have a stepped or other configuration having a reduced diameter distal region 630, as shown in FIG. 6B, which shows an enlarged view of the distal region 630 of the sheath 605. The distal region 630 of the sheath can be sized for insertion into the carotid artery, typically having an inner diameter in the range from 2.16 mm (0.085 inch) to 2.92 mm (0.115 inch) with the remaining proximal region of the sheath having larger outside and luminal diameters, with the inner diameter typically being in the range from 2.794 mm (0.110 inch) to 3.43 mm (0.135 inch). The larger luminal diameter of the proximal region minimizes the overall flow resistance of the sheath. In an embodiment, the reduced-diameter distal section 630 has a length of approximately 2 cm to 4 cm. The relatively short length of the reduced-diameter distal section 630 permits this section to be positioned in the common carotid artery CCA via the transcarotid approach with reduced risk that the distal end of the sheath 605 will contact the bifurcation B. Moreover, the reduced diameter section 630 also permits a reduction in size of the arteriotomy for introducing the sheath 605 into the artery while having a minimal impact in the level of flow resistance.

Further, the reduced distal diameter section may be more flexible and thus more conformal to the lumen of the vessel.

With reference again to FIG. 6A, the proximal extension 610, which is an elongated body, has an inner lumen which is contiguous with an inner lumen of the sheath 605. The lumens can be joined by the Y-connector 620 which also connects a lumen of the flow line 615 to the sheath. In the assembled system, the flow line 615 connects to and forms a first leg of the retrograde shunt 120 (FIG. 1). The proximal extension 610 can have a length sufficient to space the hemostasis valve 625 well away from the Y-connector 620, which is adjacent to the percutaneous or surgical insertion site. By spacing the hemostasis valve 625 away from a percutaneous insertion site, the physician can introduce a stent delivery system or other working catheter into the proximal extension 610 and sheath 605 while staying out of the fluoroscopic field when fluoroscopy is being performed. In an embodiment, the proximal extension is about 16.9 cm from a distal most junction (such as at the hemostasis valve) with the sheath 605 to the proximal end of the proximal extension. In an embodiment, the proximal extension has an inner diameter of 0.125 inch and an outer diameter of 0.175 inch. In an embodiment, the proximal extension has a wall thickness of 0.025 inch. The inner diameter may range, for example, from 0.60 inch to 0.150 inch with a wall thickness of 0.010 inch to 0.050 inch. In another embodiment, the inner diameter may range, for example, from 0.150 inch to 0.250 inch with a wall thickness of 0.025 inch to 0.100 inch. The dimensions of the proximal extension may vary. In an embodiment, the proximal extension has a length within the range of about 12-20 cm. In another embodiment, the proximal extension has a length within the range of about 20-30 cm.

In an embodiment, the distance along the sheath from the hemostasis valve 625 to the distal tip of the sheath 605 is in the range of about 25 and 40 cm. In an embodiment, the distance is in the range of about 30 and 35 cm. With a system configuration that allows 2.5 cm of sheath introduction into the artery, and an arterial distance of between 5 and 10 cm from the arteriotomy site to the target site, this system enables a distance in the range of about 32.5 cm to 42.5 cm from the hemostasis valve 625 (the location of interventional device introduction into the access sheath) to the target site of between 32 and 43 cm. This distance is about a third the distance required in prior art technology.

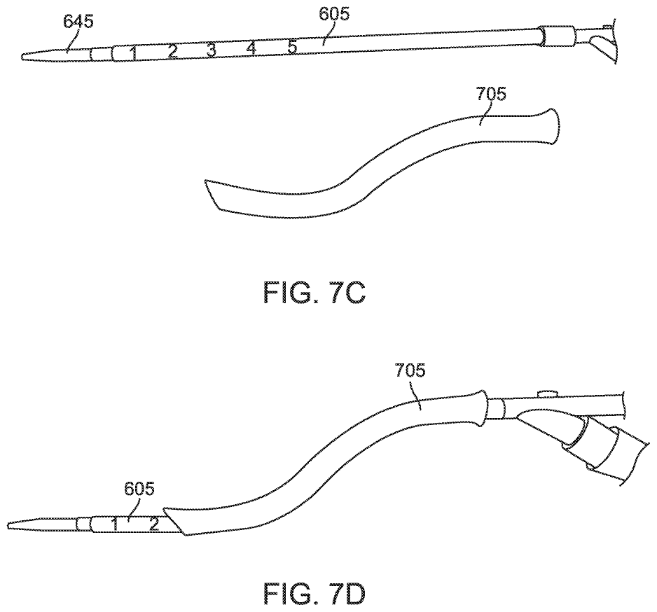

A flush line 635 can be connected to the side of the hemostasis valve 625 and can have a stopcock 640 at its proximal or remote end. The flush-line 635 allows for the introduction of saline, contrast fluid, or the like, during the procedures. The flush line 635 can also allow pressure monitoring during the procedure. A dilator 645 having a tapered distal end 650 can be provided to facilitate introduction of the distal sheath 605 into the common carotid artery. The dilator 645 can be introduced through the hemostasis valve 625 so that the tapered distal end 650 extends through the distal end of the sheath 605, as best seen in FIG. 7A. The dilator 645 can have a central lumen to accommodate a guide wire.

Typically, the guide wire is placed first into the vessel, and the dilator/sheath combination travels over the guide wire as it is being introduced into the vessel.

Optionally, a sheath stopper 705 such as in the form of a tube may be provided which is coaxially received over the exterior of the distal sheath 605, also as seen in FIG. 7A. The sheath stopper 705 is configured to act as a sheath stopper to prevent the sheath from being inserted too far into the vessel. The sheath stopper 705 is sized and shaped to be positioned over the sheath body 605 such that it covers a portion of the sheath body 605 and leaves a distal portion of the sheath body 605 exposed. The sheath stopper 705 may have a flared proximal end 710 that engages the adapter 620, and a distal end 715. Optionally, the distal end 715 may be beveled, as shown in FIG. 7B. The sheath stopper 705 may serve at least two purposes. First, the length of the sheath stopper 705 limits the introduction of the sheath 605 to the exposed distal portion of the sheath 605, as seen in FIG. 7A, such that the sheath insertion length is limited to the exposed distal portion of the sheath. In an embodiment, the sheath stopper limits the exposed distal portion to a range between 2 and 3 cm. In an embodiment, the sheath stopper limited the exposed distal portion to 2.5 cm. In other words, the sheath stopper may limit insertion of the sheath into the artery to a range between about 2 and 3 cm or to 2.5 cm. Second, the sheath stopper 705 can engage a pre-deployed puncture closure device disposed in the carotid artery wall, if present, to permit the sheath 605 to be withdrawn without dislodging the closure device. The sheath stopper 705 may be manufactured from clear material so that the sheath body may be clearly visible underneath the sheath stopper 705. The sheath stopper 705 may also be made from flexible material, or the sheath stopper 705 include articulating or sections of increased flexibility so that it allows the sheath to bend as needed in a proper position once inserted into the artery.

The sheath stopper may be plastically bendable such that it can be bent into a desired shape such that it retains the shape when released by a user. The distal portion of the sheath stopper may be made from stiffer material, and the proximal portion may be made from more flexible material. In an embodiment, the stiffer material is 85A durometer and the more flexible section is 50A durometer. In an embodiment, the stiffer distal portion is 1 to 4 cm of the sheath stopper 705. The sheath stopper 705 may be removable from the sheath so that if the user desired a greater length of sheath insertion, the user could remove the sheath stopper 705, cut the length (of the sheath stopper) shorter, and re-assemble the sheath stopper 705 onto the sheath such that a greater length of insertable sheath length protrudes from the sheath stopper 705.

FIG. 7C shows another embodiment of a sheath stopper 705 positioned adjacent a sheath 605 with a dilator 645 positioned therein. The sheath stopper 705 of FIG. 7C may be deformed from a first shaped, such as a straight shape, into a second different from the first shape wherein the sheath stopper retains the second shape until a sufficient external force acts on the sheath stopper to change its shape. The second shape may be for example non-straight, curved, or an otherwise contoured or irregular shape. For example, FIG. 7C shows the sheath stopper 705 having multiple bends as well as straight sections.

FIG. 7C shows just an example and it should be appreciated that the sheath stopper 705 may be shaped to have any quantity of bends along its longitudinal axis. FIG. 7D shows the sheath stopper 705 positioned on the sheath 605. The sheath stopper 705 has a greater stiffness than the sheath 605 such that the sheath 605 takes on a shape or contour that conforms to the shape of contour of the sheath stopper 705.

The sheath stopper 705 may be shaped according to an angle of the sheath insertion into the artery and the depth of the artery or body habitus of the patient. This feature reduces the force of the sheath tip in the blood vessel wall, especially in cases where the sheath is inserted at a steep angle into the vessel. The sheath stopper may be bent or otherwise deformed into a shape that assists in orienting the sheath coaxially with the artery being entered even if the angle of the entry into the arterial incision is relatively steep. The sheath stopper may be shaped by an operator prior to sheath insertion into the patient. Or, the sheath stopper may be shaped and/or re-shaped in situ after the sheath has been inserted into the artery.

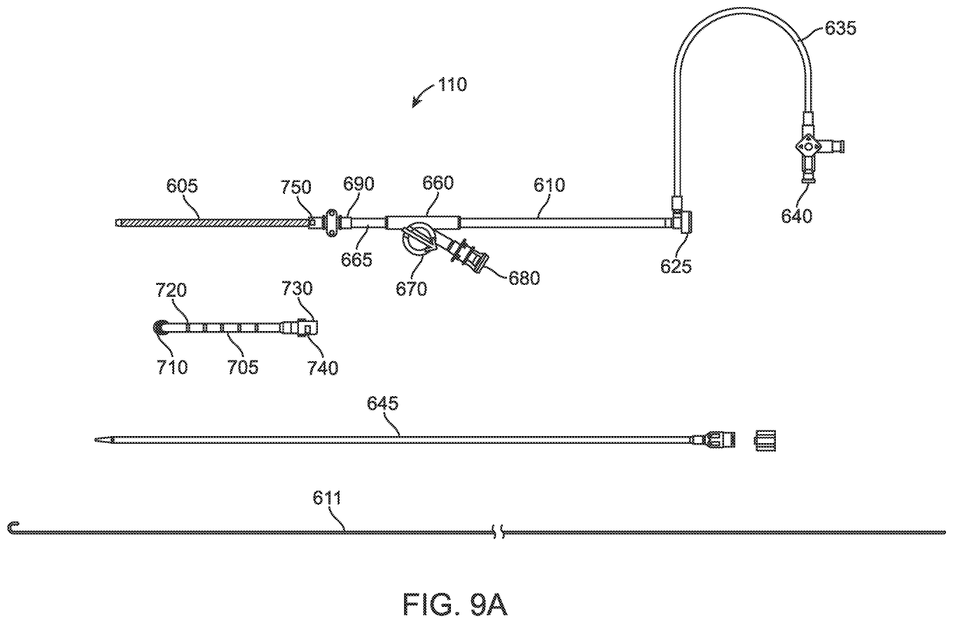

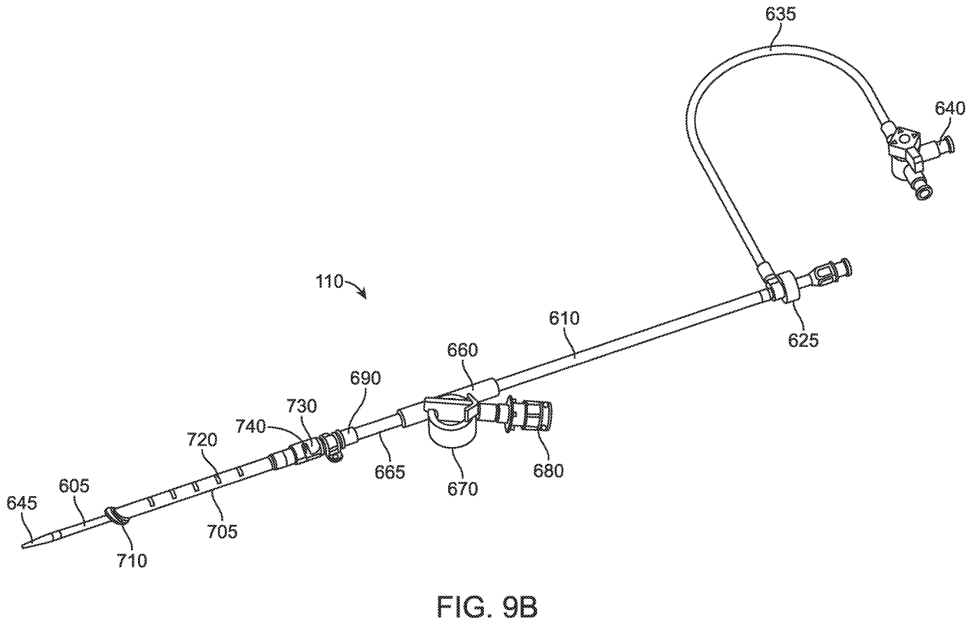

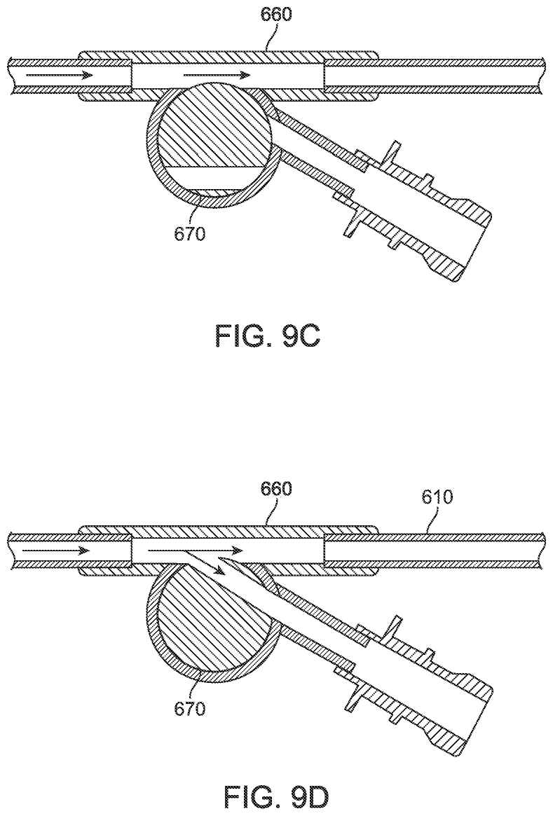

In another embodiment, as shown in FIG. 9A, the sheath stopper 705 includes a distal base or flange 710 sized and shaped to distribute the force of the sheath stopper over a larger area on the vessel wall and thereby reduce the risk of vessel injury or accidental insertion of the sheath stopper through the arteriotomy and into the vessel. The flange 710 may have a rounded shape or other atraumatic shape that is sufficiently large to distribute the force of the sheath stopper over a large area on the vessel wall. In an embodiment, the flange is inflatable or mechanically expandable. For example, the arterial sheath and sheath stopper may be inserted through a small puncture in the skin into the surgical area, and then expanded prior to insertion of the sheath into the artery.

The sheath stopper may include one or more cutouts or indents 720 along the length of the sheath stopper which are patterned in a staggered configuration such that the indents increase the bendability of the sheath stopper while maintaining axial strength to allow forward force of the sheath stopper against the arterial wall. The indents may also be used to facilitate securement of the sheath to the patient via sutures, to mitigate against sheath dislodgement. The sheath stopper may also include a connector element 730 on the proximal end which corresponds to features on the arterial sheath such that the sheath stopper can be locked or unlocked from the arterial sheath. For example, the connector element is a hub with generally L-shaped slots 740 that correspond to pins 750 on the hub to create a bayonet mount-style connection. In this manner, the sheath stopper can be securely attached to the hub to reduce the likelihood that the sheath stopper will be inadvertently removed from the hub unless it is unlocked from the hub.

The distal sheath 605 can be configured to establish a curved transition from a generally anterior-posterior approach over the common carotid artery to a generally axial luminal direction within the common carotid artery. Arterial access through the common carotid arterial wall either from a direct surgical cut down or a percutaneous access may require an angle of access that is typically larger than other sites of arterial access. This is due to the fact that the common carotid insertion site is much closer to the treatment site (i.e., carotid bifurcation) than from other access points. A larger access angle is needed to increase the distance from the insertion site to the treatment site to allow the sheath to be inserted at an adequate distance without the sheath distal tip reaching the carotid bifurcation. For example, the sheath insertion angle is typically 30-45 degrees or even larger via a transcarotid access, whereas the sheath insertion angle may be 15-20 degrees for access into a femoral artery.

Thus the sheath must take a greater bend than is typical with introducer sheaths, without kinking and without causing undue force on the opposing arterial wall. In addition, the sheath tip desirably does not be abut or contact the arterial wall after insertion in a manner that would restrict flow into the sheath. The sheath insertion angle is defined as the angle between the luminal axis of the artery and the longitudinal axis of the sheath.

Another sheath configuration comprises a curved dilator inserted into a straight but flexible sheath, so that the dilator and sheath are curved during insertion. The sheath is flexible enough to conform to the anatomy after dilator removal.

In an embodiment, the sheath has built-in puncturing capability and atraumatic tip analogous to a guide wire tip. This eliminates the need for needle and wire exchange currently used for arterial access according to the micropuncture technique, and can thus save time, reduce blood loss, and require less surgeon skill.

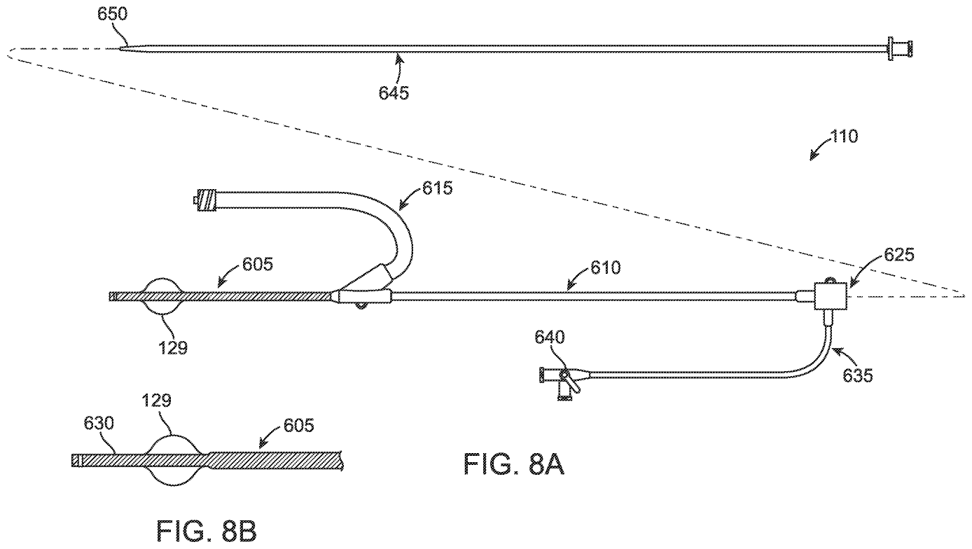

FIG. 8A shows another embodiment of the arterial access device 110. This embodiment is substantially the same as the embodiment shown in FIG. 6A, except that the distal sheath 605 includes an occlusion element 129 for occluding flow through, for example the common carotid artery. If the occluding element 129 is an inflatable structure such as a balloon or the like, the sheath 605 can include an inflation lumen that communicates with the occlusion element 129. The occlusion element 129 can be an inflatable balloon, but it could also be an inflatable cuff, a conical or other circumferential element which flares outwardly to engage the interior wall of the common carotid artery to block flow therepast, a membrane-covered braid, a slotted tube that radially enlarges when axially compressed, or similar structure which can be deployed by mechanical means, or the like. In the case of balloon occlusion, the balloon can be compliant, non-compliant, elastomeric, reinforced, or have a variety of other characteristics. In an embodiment, the balloon is an elastomeric balloon which is closely received over the exterior of the distal end of the sheath prior to inflation.

When inflated, the elastomeric balloon can expand and conform to the inner wall of the common carotid artery. In an embodiment, the elastomeric balloon is able to expand to a diameter at least twice that of the non-deployed configuration, frequently being able to be deployed to a diameter at least three times that of the undeployed configuration, more preferably being at least four times that of the undeployed configuration, or larger.

As shown in FIG. 8B, the distal sheath 605 with the occlusion element 129 can have a stepped or other configuration having a reduced diameter distal region 630. The distal region 630 can be sized for insertion into the carotid artery with the remaining proximal region of the sheath 605 having larger outside and luminal diameters, with the inner diameter typically being in the range from 2.794 mm (0.110 inch) to 3.43 mm (0.135 inch). The larger luminal diameter of the proximal region minimizes the overall flow resistance of the sheath. In an embodiment, the reduced-diameter distal section 630 has a length of approximately 2 cm to 4 cm. The relatively short length of the reduced-diameter distal section 630 permits this section to be positioned in the common carotid artery CCA via the transcarotid approach with reduced risk that the distal end of the sheath 605 will contact the bifurcation B.

In a situation with a sharp sheath insertion angle and/or a short length of sheath inserted in the artery, such as one might see in a transcarotid access procedure, the distal tip of the sheath has a higher likelihood of being partially or totally positioned against the vessel wall, thereby restricting flow into the sheath. In an embodiment, the sheath is configured to center the tip in the lumen of the vessel. One such embodiment includes a balloon such as the occlusion element 129 described above. In another embodiment, a balloon may not be occlusive to flow but still center the tip of the sheath away from a vessel wall, like an inflatable bumper. In another embodiment, expandable features are situated at the tip of the sheath and mechanically expanded once the sheath is in place. Examples of mechanically expandable features include braided structures or helical structures or longitudinal struts which expand radially when shortened.

In an embodiment, occlusion of the vessel proximal to the distal tip of the sheath may be done from the outside of the vessel, as in a Rumel tourniquet or vessel loop proximal to sheath insertion site. In an alternate embodiment, an occlusion device may fit externally to the vessel around the sheath tip, for example an elastic loop, inflatable cuff, or a mechanical clamp that could be tightened around the vessel and distal sheath tip. In a system of flow reversal, this method of vessel occlusion minimizes the area of static blood flow, thereby reducing risk of thrombus formation, and also ensure that the sheath tip is axially aligned with vessel and not partially or fully blocked by the vessel wall.

Another arterial access device is shown in FIGS. 9A-9D . This configuration has a different style of connection to the flow shunt than the versions described previously. FIG. 9A shows the components of the arterial access device 110 including arterial access sheath 605, sheath dilator 645, sheath stopper 705, and sheath guidewire 611. FIG. 9B shows the arterial access device 110 as it would be assembled for insertion over the sheath guide wire 611 into the carotid artery. After the sheath is inserted into the artery and during the procedure, the sheath guide wire 611 and sheath dilator 705 are removed. In this configuration, the sheath has a sheath body 605, proximal extension 610, and proximal hemostasis valve 625 with flush line 635 and stopcock 640. The proximal extension 610 extends from a Y-adapter 660 to the hemostasis valve 625 where the flush line 635 is connected. The sheath body 605 is the portion that is sized to be inserted into the carotid artery and is actually inserted into the artery during use.

Instead of a Y-connector with a flow line connection terminating in a valve, the sheath has a Y-adaptor 660 that connects the distal portion of the sheath to the proximal extension 610. The Y-adapter can also include a valve 670 that can be operated to open and close fluid connection to a connector or hub 680 that can be removably connected to a flow line such as a shunt. The valve 670 is positioned immediately adjacent to an internal lumen of the adapter 660, which communicates with the internal lumen of the sheath body 605.

FIGS. 9C and 9D show details in cross section of the Y-adaptor 660 with the valve 670 and the hub 680. FIG. 9C shows the valve closed to the connector. This is the position that the valve would be in during prep of the arterial sheath. The valve is configured so that there is no potential for trapped air during prep of the sheath. FIG. 9D shows the valve open to the connector. This position would be used once the flow shunt 120 is connected to hub 680, and would allow blood flow from the arterial sheath into the shunt. This configuration eliminates the need to prep both a flush line and flow line, instead allowing prep from the single flush line 635 and stopcock 640. This single-point prep is identical to prep of conventional introducer sheaths which do not have connections to shunt lines, and is therefore more familiar and convenient to the user. In addition, the lack of flow line on the sheath makes handling of the arterial sheath easier during prep and insertion into the artery.

With reference again to FIG. 9A, the sheath may also contain a second more distal connector 690, which is separated from the Y-adaptor 660 by a segment of tubing 665. A purpose of this second connector and the tubing 665 is to allow the valve 670 to be positioned further proximal from the distal tip of the sheath, while still limiting the length of the insertable portion of the sheath 605, and therefore allow a reduced level of exposure of the user to the radiation source as the flow shunt is connected to the arterial sheath during the procedure. In an embodiment, the distal connector 690 contains suture eyelets to aid in securement of the sheath to the patient once positioned.

During a transcarotid artery revascularization (TCAR) procedure, the arterial sheath 605 can be inserted into the common carotid artery (CCA) of the patient. As described elsewhere herein, to achieve reverse flow of blood, the CCA may be occluded to stop antegrade blood flow from the aorta through the CCA. Flow through the CCA can be occluded with an external vessel loop or tape, a vascular clamp, an internal occlusion member such as a balloon, or other type of occlusion means. When flow through CCA is blocked, the natural pressure gradient between the internal carotid artery (ICA) and the venous system will cause blood to flow in a retrograde or reverse direction from the cerebral vasculature. Blood from the ICA and the external carotid artery (ECA) flows in a retrograde direction and the systems described herein allow the retrograde blood to flow into the sheath 605, through the flow controller 1130, the venous sheath 910, and then returned into the patient's femoral vein as described elsewhere herein. Loose embolic material can be carried with the retrograde blood flow into the arterial sheath 605.

### Venous Return Device

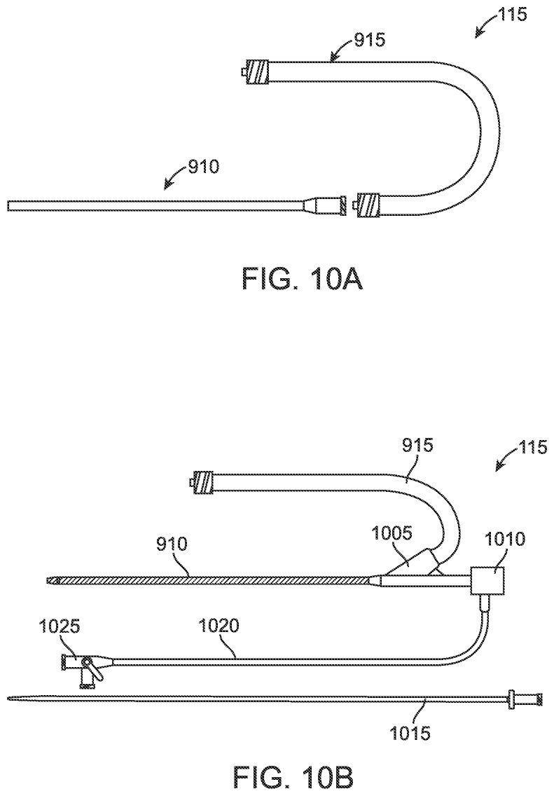

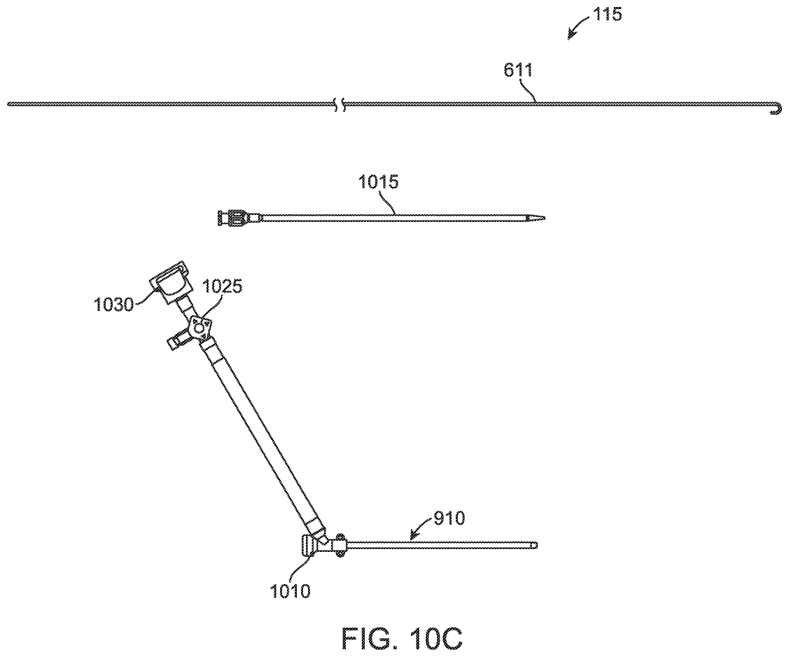

Referring now to FIGS. 10A and 10B, the venous return device 115 can comprise a distal sheath 910 and a flow line 915, which connects to and forms a leg of the shunt 120 when the system is in use. The distal sheath 910 is adapted to be introduced through an incision or puncture into a venous return location, such as the jugular vein or femoral vein. The distal sheath 910 and flow line 915 can be permanently affixed, or can be attached using a conventional luer fitting, as shown in FIG. 10A. Optionally, as shown in FIG. 10B, the sheath 910 can be joined to the flow line 915 by a Y-connector 1005. The Y-connector 1005 can include a hemostasis valve 1010. The venous return device also comprises a venous sheath dilator 1015 and an introducer guide wire 611 to facilitate introduction of the venous return device into the internal jugular vein or other vein. As with the arterial access dilator 645, the venous dilator 1015 includes a central guide wire lumen so the venous sheath and dilator combination can be placed over the guide wire 611.

Optionally, the venous sheath 910 can include a flush line 1020 with a stopcock 1025 at its proximal or remote end.

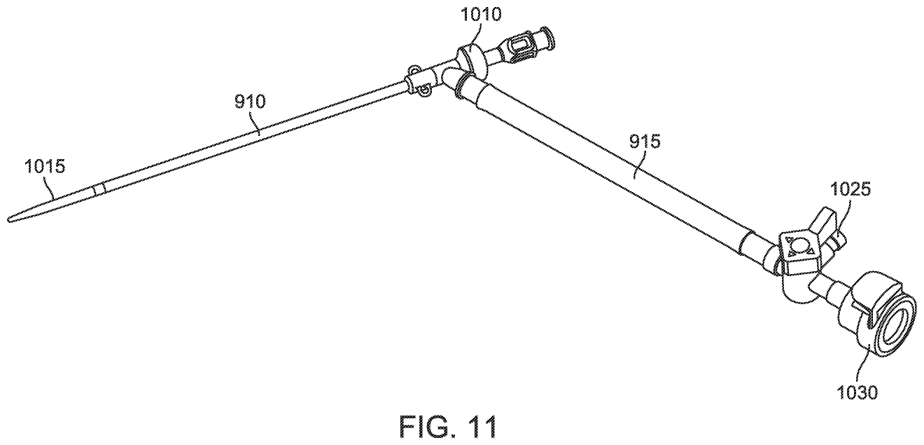

An alternate configuration is shown in FIGS. 10C and 11. FIG. 10C shows the components of the venous return device 115 including venous return sheath 910, sheath dilator 1015, and sheath guidewire 611. FIG. 11 shows the venous return device 115 as it would be assembled for insertion over the sheath guide wire 611 into a central vein.

Once the sheath is inserted into the vein, the dilator and guidewire are removed. The venous sheath can include a hemostasis valve 1010 and flow line 915. A stopcock 1025 on the end of the flow line allows the venous sheath to be flushed via the flow line prior to use. This configuration allows the sheath to be prepped from a single point, as is done with conventional introducer sheaths. Connection to the flow shunt 120 is made with a connector 1030 on the stopcock 1025.

In order to reduce the overall system flow resistance, the arterial access flow line 615 (FIG. 6A) and the venous return flow line 915, and Y-connectors 620 (FIGS. 6A) and 1005, can each have a relatively large flow lumen inner diameter, typically being in the range from 2.54 mm (0.100 inch) to 5.08 mm (0.200 inch), and a relatively short length, typically being in the range from 10 cm to 20 cm. The low system flow resistance is desirable since it permits the flow to be maximized during portions of a procedure when the risk of emboli is at its greatest. The low system flow resistance also allows the use of a variable flow resistance for controlling flow in the system, as described in more detail below. The dimensions of the venous return sheath 910 can be generally the same as those described for the arterial access sheath 605 above. In the venous return sheath, an extension for the hemostasis valve 1010 is not required.

### Retrograde Shunt

The shunt 120 can be formed of a single tube or multiple, connected tubes that provide fluid communication between the arterial access catheter 110 and the venous return catheter 115 to provide a pathway for retrograde blood flow therebetween. As shown in FIG. 1A, the shunt 120 connects at one end (via connector 127a) to the flow line 615 of the arterial access device 110, and at an opposite end (via connector 127b) to the flow line 915 of the venous return catheter 115.

In an embodiment, the shunt 120 can be formed of at least one tube that communicates with the flow control assembly 125. The shunt 120 can be any structure that provides a fluid pathway for blood flow. The shunt 120 can have a single lumen or it can have multiple lumens. The shunt 120 can be removably attached to the flow control assembly 125, arterial access device 110, and/or venous return device 115. Prior to use, the user can select a shunt 120 with a length that is most appropriate for use with the arterial access location and venous return location. In an embodiment, the shunt 120 can include one or more extension tubes that can be used to vary the length of the shunt 120. The extension tubes can be modularly attached to the shunt 120 to achieve a desired length. The modular aspect of the shunt 120 permits the user to lengthen the shunt 120 as needed depending on the site of venous return. For example, in some patients, the internal jugular vein IJV is small and/or tortuous. The risk of complications at this site may be higher than at some other locations, due to proximity to other anatomic structures. In addition, hematoma in the neck may lead to airway obstruction and/or cerebral vascular complications. Consequently, for such patients it may be desirable to locate the venous return site at a location other than the internal jugular vein IJV, such as the femoral vein. A femoral vein return site may be accomplished percutaneously, with lower risk of serious complication, and also offers an alternative venous access to the central vein if the internal jugular vein IJV is not available.

Furthermore, the femoral venous return changes the layout of the reverse flow shunt such that the shunt controls may be located closer to the “working area” of the intervention, where the devices are being introduced and the contrast injection port is located.

In an embodiment, the shunt 120 has an internal diameter of 4.76 mm ( 3/16 inch) and has a length of 40-70 cm. As mentioned, the length of the shunt can be adjusted.

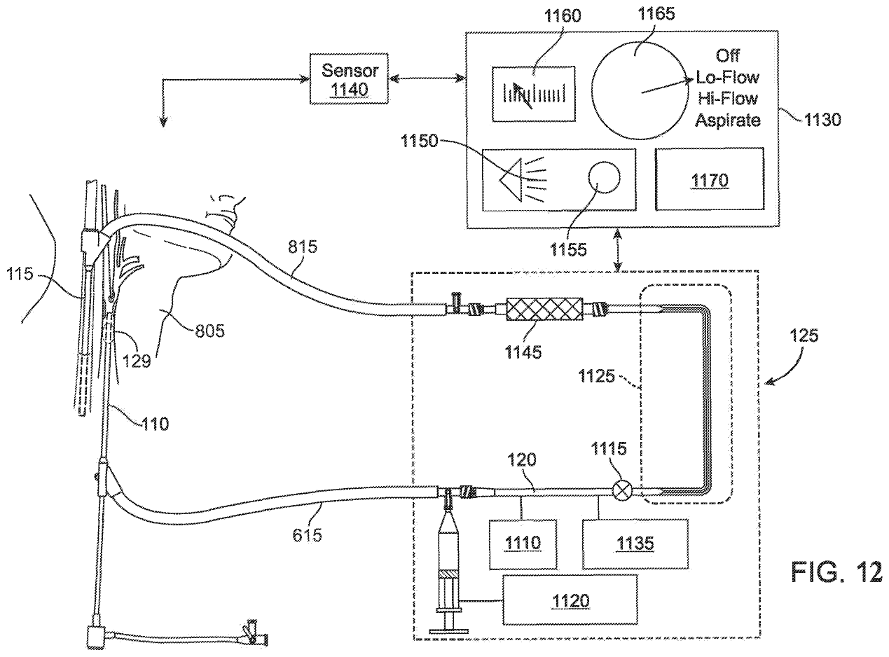

In an embodiment, connectors between the shunt and the arterial and/or venous access devices are configured to minimize flow resistance. In an embodiment, the arterial access sheath 110, the retrograde shunt 120, and the venous return sheath 115 are combined to create a low flow resistance arterio-venous AV shunt, as shown in FIGS. 1A-1D. As described above, the connections and flow lines of all these devices are optimized to minimize or reduce the resistance to flow. In an embodiment, the AV shunt has a flow resistance which enables a flow of up to 300 mL/minute when no device is in the arterial sheath 110 and when the AV shunt is connected to a fluid source with the viscosity of blood and a static pressure head of 60 mmHg. The actual shunt resistance may vary depending on the presence or absence of a check valve 1115 or a filter 1145 (as shown in FIG. 12), or the length of the shunt, and may enable a flow of between 150 and 300 mL/min.

When there is a device such as a stent delivery catheter in the arterial sheath, there is a section of the arterial sheath that has increased flow resistance, which in turn increases the flow resistance of the overall AV shunt. This increase in flow resistance has a corresponding reduction in flow. In an embodiment, the Y-arm 620 as shown in FIG. 6A connects the arterial sheath body 605 to the flow line 615 some distance away from the hemostasis valve 625 where the catheter is introduced into the sheath. This distance is set by the length of the proximal extension 610. Thus the section of the arterial sheath that is restricted by the catheter is limited to the length of the sheath body 605. The actual flow restriction will depend on the length and inner diameter of the sheath body 605, and the outer diameter of the catheter. As described above, the sheath length may range from 5 to 15 cm, usually being from 10 cm to 12 cm, and the inner diameter is typically in the range from 7 Fr (1 Fr=0.33 mm), to 10 Fr, usually being 8 Fr. Stent delivery catheters may range from 3.7 Fr. to 5.0 or 6.0 Fr, depending on the size of the stent and the manufacturer. This restriction may further be reduced if the sheath body is designed to increase in inner diameter for the portion not in the vessel (a stepped sheath body), as shown in FIG. 6B. Since flow restriction is proportional to luminal distances to the fourth power, small increases in luminal or annular areas result in large reductions in flow resistance.

Actual flow through the AV shunt when in use will further depend on the cerebral blood pressures and flow resistances of the patient.

### Flow Control Assembly-Regulation and Monitoring of Retrograde Flow

The flow control assembly 125 interacts with the retrograde shunt 120 to regulate and/or monitor the retrograde flow rate from the common carotid artery to the venous return site, such as the femoral vein, internal jugular vein, or to the external receptacle 130. In this regard, the flow control assembly 125 enables the user to achieve higher maximum flow rates than existing systems and to also selectively adjust, set, or otherwise modulate the retrograde flow rate. Various mechanisms can be used to regulate the retrograde flow rate. The flow control assembly 125 enables the user to configure retrograde blood flow in a manner that is suited for various treatment regimens, as described below.

FIG. 12 shows an example of the system 100 with a schematic representation of the flow control assembly 125, which is positioned along the shunt 120 such that retrograde blood flow passes through or otherwise communicates with at least a portion of the flow control assembly 125. The flow control assembly 125 can include various controllable mechanisms for regulating and/or monitoring retrograde flow. The mechanisms can include various means of controlling the retrograde flow, including one or more pumps 1110, valves 1115, syringes 1120 and/or a variable resistance component 1125. The flow control assembly 125 can be manually controlled by a user and/or automatically controlled via a controller 1130 to vary the flow through the shunt 120. For example, by varying the flow resistance, the rate of retrograde blood flow through the shunt 120 can be controlled.

The controller 1130, which is described in more detail below, can be integrated into the flow control assembly 125 or it can be a separate component that communicates with the components of the flow control assembly 125.

In addition, the flow control assembly 125 can include one or more flow sensors 1135 and/or anatomical data sensors 1140 (described in detail below) for sensing one or more aspects of the retrograde flow. A filter 1145 can be positioned along the shunt 120 for removing emboli before the blood is returned to the venous return site. When the filter 1145 is positioned upstream of the controller 1130, the filter 1145 can prevent emboli from entering the controller 1145 and potentially clogging the variable flow resistance component 1125. It should be appreciated that the various components of the flow control assembly 125 (including the pump 1110, valves 1115, syringes 1120, variable resistance component 1125, sensors 1135/1140, and filter 1145) can be positioned at various locations along the shunt 120 and at various upstream or downstream locations relative to one another. The components of the flow control assembly 125 are not limited to the locations shown in FIG. 12. Moreover, the flow control assembly 125 does not necessarily include all of the components but can rather include various sub-combinations of the components. For example, a syringe could optionally be used within the flow control assembly 125 for purposes of regulating flow or it could be used outside of the assembly for purposes other than flow regulation, such as to introduce fluid such as radiopaque contrast into the artery in an antegrade direction via the shunt 120.

Both the variable resistance component 1125 and the pump 1110 can be coupled to the shunt 120 to control the retrograde flow rate. The variable resistance component 1125 controls the flow resistance, while the pump 1110 provides for positive displacement of the blood through the shunt 120. Thus, the pump can be activated to drive the retrograde flow rather than relying on the perfusion stump pressures of the ECA and ICA and the venous back pressure to drive the retrograde flow. The pump 1110 can be a peristaltic tube pump or any type of pump including a positive displacement pump. The pump 1110 can be activated and deactivated (either manually or automatically via the controller 1130) to selectively achieve blood displacement through the shunt 120 and to control the flow rate through the shunt 120. Displacement of the blood through the shunt 120 can also be achieved in other manners including using the aspiration syringe 1120, or a suction source such as a vacutainer, vaculock syringe, or wall suction may be used. The pump 1110 can communicate with the controller 1130.

One or more flow control valves 1115 can be positioned along the pathway of the shunt. The valve(s) can be manually actuated or automatically actuated (via the controller 1130). The flow control valves 1115 can be, for example one-way valves to prevent flow in the antegrade direction in the shunt 120, check valves, or high pressure valves which would close off the shunt 120, for example during high-pressure contrast injections (which are intended to enter the arterial vasculature in an antegrade direction). In an embodiment, the one-way valves are low flow-resistance valves for example that described in U.S. Pat. No. 5,727,594, or other low resistance valves.

In an embodiment of a shunt with both a filter 1145 and a one-way check valve 1115, the check valve is located downstream of the filter. In this manner, if there is debris traveling in the shunt, it is trapped in the filter before it reaches the check valve. Many check valve configurations include a sealing member that seals against a housing that contains a flow lumen. Debris may have the potential to be trapped between the sealing member and the housing, thus compromising the ability of the valve to seal against backwards pressure.

The controller 1130 communicates with components of the system 100 including the flow control assembly 125 to enable manual and/or automatic regulation and/or monitoring of the retrograde flow through the components of the system 100 (including, for example, the shunt 120, the arterial access device 110, the venous return device 115 and the flow control assembly 125). For example, a user can actuate one or more actuators on the controller 1130 to manually control the components of the flow control assembly 125.

Manual controls can include switches or dials or similar components located directly on the controller 1130 or components located remote from the controller 1130 such as a foot pedal or similar device. The controller 1130 can also automatically control the components of the system 100 without requiring input from the user. In an embodiment, the user can program software in the controller 1130 to enable such automatic control. The controller 1130 can control actuation of the mechanical portions of the flow control assembly 125. The controller 1130 can include circuitry or programming that interprets signals generated by sensors 1135/1140 such that the controller 1130 can control actuation of the flow control assembly 125 in response to such signals generated by the sensors.

The representation of the controller 1130 in FIG. 12 is merely exemplary. It should be appreciated that the controller 1130 can vary in appearance and structure. The controller 1130 is shown in FIG. 12 as being integrated in a single housing. This permits the user to control the flow control assembly 125 from a single location. It should be appreciated that any of the components of the controller 1130 can be separated into separate housings. Further, FIG. 12 shows the controller 1130 and flow control assembly 125 as separate housings. It should be appreciated that the controller 1130 and flow control regulator 125 can be integrated into a single housing or can be divided into multiple housings or components.

### Flow State Indicator(s)

The controller 1130 can include one or more indicators that provides a visual and/or audio signal to the user regarding the state of the retrograde flow. An audio indication advantageously reminds the user of a flow state without requiring the user to visually check the flow controller 1130. The indicator(s) can include a speaker 1150 and/or a light 1155 or any other means for communicating the state of retrograde flow to the user. The controller 1130 can communicate with one or more sensors of the system to control activation of the indicator. Or, activation of the indicator can be tied directly to the user actuating one of the flow control actuators 1165. The indicator need not be a speaker or a light. The indicator could simply be a button or switch that visually indicates the state of the retrograde flow. For example, the button being in a certain state (such as a pressed or down state) may be a visual indication that the retrograde flow is in a high state. Or, a switch or dial pointing toward a particular labeled flow state may be a visual indication that the retrograde flow is in the labeled state.

### Flow Rate Actuators

The controller 1130 can include one or more actuators that the user can press, switch, manipulate, or otherwise actuate to regulate the retrograde flow rate and/or to monitor the flow rate. For example, the controller 1130 can include a flow control actuator 1165 (such as one or more buttons, knobs, dials, switches, etc.) that the user can actuate to cause the controller to selectively vary an aspect of the reverse flow. For example, in the illustrated embodiment, the flow control actuator 1165 is a knob that can be turned to various discrete positions each of which corresponds to the controller 1130 causing the system 100 to achieve a particular retrograde flow state. The states include, for example, (a) OFF; (b) LO-FLOW; (c) HI-FLOW; and (d) ASPIRATE. It should be appreciated that the foregoing states are merely exemplary and that different states or combinations of states can be used. The controller 1130 achieves the various retrograde flow states by interacting with one or more components of the system, including the sensor(s), valve(s), variable resistance component, and/or pump(s). It should be appreciated that the controller 1130 can also include circuitry and software that regulates the retrograde flow rate and/or monitors the flow rate such that the user wouldn't need to actively actuate the controller 1130.

The OFF state corresponds to a state where there is no retrograde blood flow through the shunt 120. When the user sets the flow control actuator 1165 to OFF, the controller 1130 causes the retrograde flow to cease, such as by shutting off valves or closing a stop cock in the shunt 120. The LO-FLOW and HI-FLOW states correspond to a low retrograde flow rate and a high retrograde flow rate, respectively. When the user sets the flow control actuator 1165 to LO-FLOW or HI-FLOW, the controller 1130 interacts with components of the flow control regulator 125 including pump(s) 1110, valve(s) 1115 and/or variable resistance component 1125 to increase or decrease the flow rate accordingly.

Finally, the ASPIRATE state corresponds to opening the circuit to a suction source, for example a vacutainer or suction unit, if active retrograde flow is desired.

The system can be used to vary the blood flow between various states including an active state, a passive state, an aspiration state, and an off state. The active state corresponds to the system using a means that actively drives retrograde blood flow. Such active means can include, for example, a pump, syringe, vacuum source, etc. The passive state corresponds to when retrograde blood flow is driven by the perfusion stump pressures of the ECA and ICA and possibly the venous pressure. The aspiration state corresponds to the system using a suction source, for example a vacutainer or suction unit, to drive retrograde blood flow. The off state corresponds to the system having zero retrograde blood flow such as the result of closing a stopcock or valve. The low and high flow rates can be either passive or active flow states. In an embodiment, the particular value (such as in ml/min) of either the low flow rate and/or the high flow rate can be predetermined and/or pre-programmed into the controller such that the user does not actually set or input the value. Rather, the user simply selects “high flow” and/or “low flow” (such as by pressing an actuator such as a button on the controller 1130) and the controller 1130 interacts with one or more of the components of the flow control assembly 125 to cause the flow rate to achieve the predetermined high or low flow rate value. In another embodiment, the user sets or inputs a value for low flow rate and/or high flow rate such as into the controller. In another embodiment, the low flow rate and/or high flow rate is not actually set. Rather, external data (such as data from the anatomical data sensor 1140) is used as the basis for affects the flow rate.

The flow control actuator 1165 can be multiple actuators, for example one actuator, such as a button or switch, to switch state from LO-FLOW to HI-FLOW and another to close the flow loop to OFF, for example during a contrast injection where the contrast is directed antegrade into the carotid artery. In an embodiment, the flow control actuator 1165 can include multiple actuators. For example, one actuator can be operated to switch flow rate from low to high, another actuator can be operated to temporarily stop flow, and a third actuator (such as a stopcock) can be operated for aspiration using a syringe. In another example, one actuator is operated to switch to LO-FLOW and another actuator is operated to switch to HI-FLOW. Or, the flow control actuator 1165 can include multiple actuators to switch states from LO-FLOW to HI-FLOW and additional actuators for fine-tuning flow rate within the high flow state and low flow state. Upon switching between LO-FLOW and HI-FLOW, these additional actuators can be used to fine-tune the flow rates within those states. Thus, it should be appreciated that within each state (i.e. high flow state and low flow states) a variety of flow rates can be dialed in and fine-tuned. A wide variety of actuators can be used to achieve control over the state of flow.

The controller 1130 or individual components of the controller 1130 can be located at various positions relative to the patient and/or relative to the other components of the system 100. For example, the flow control actuator 1165 can be located near the hemostasis valve where any interventional tools are introduced into the patient in order to facilitate access to the flow control actuator 1165 during introduction of the tools. The location may vary, for example, based on whether a transfemoral or a transcarotid approach is used, as shown in FIG. 1 A-C. The controller 1130 can have a wireless connection to the remainder of the system 100 and/or a wired connection of adjustable length to permit remote control of the system 100. The controller 1130 can have a wireless connection with the flow control regulator 125 and/or a wired connection of adjustable length to permit remote control of the flow control regulator 125. The controller 1130 can also be integrated in the flow control regulator 125. Where the controller 1130 is mechanically connected to the components of the flow control assembly 125, a tether with mechanical actuation capabilities can connect the controller 1130 to one or more of the components. In an embodiment, the controller 1130 can be positioned a sufficient distance from the system 100 to permit positioning the controller 1130 outside of a radiation field when fluoroscopy is in use.

The controller 1130 and any of its components can interact with other components of the system (such as the pump(s), sensor(s), shunt, etc.) in various manners.

For example, any of a variety of mechanical connections can be used to enable communication between the controller 1130 and the system components. Alternately, the controller 1130 can communicate electronically or magnetically with the system components. Electro-mechanical connections can also be used. The controller 1130 can be equipped with control software that enables the controller to implement control functions with the system components. The controller itself can be a mechanical, electrical or electro-mechanical device. The controller can be mechanically, pneumatically, or hydraulically actuated or electromechanically actuated (for example in the case of solenoid actuation of flow control state). The controller 1130 can include a computer, computer processor, and memory, as well as data storage capabilities.

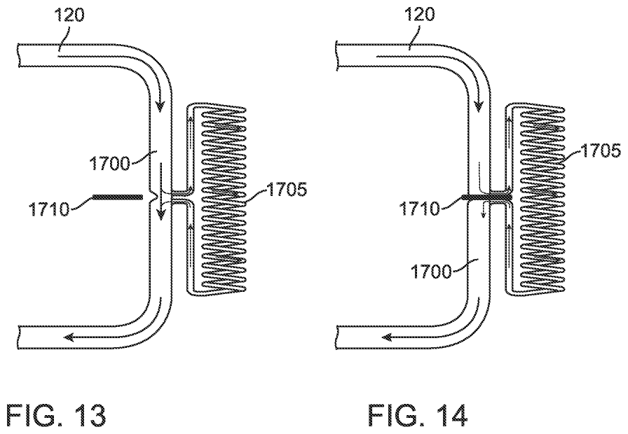

FIG. 13 shows an exemplary embodiment of a variable flow control element 1125. In this embodiment, the flow resistance through shunt 120 may be changed by providing two or more alternative flow paths to create a low and high resistance flow path.

As shown in FIG. 13, the flow through shunt 120 passes through a main lumen 1700 as well as secondary lumen 1705. The secondary lumen 1705 is longer and/or has a smaller diameter than the main lumen 1700. Thus, the secondary lumen 1705 has higher flow resistance than the main lumen 1700. By passing the blood through both these lumens, the flow resistance will be at a minimum. Blood is able to flow through both lumens 1700 and 1705 due to the pressure drop created in the main lumen 1700 across the inlet and outlet of the secondary lumen 1705. This has the benefit of preventing stagnant blood. As shown in FIG. 14, by blocking flow through the main lumen 1700 of shunt 120, the flow is diverted entirely to the secondary lumen 1705, thus increasing the flow resistance and reducing the blood flow rate. It will be appreciated that additional flow lumens could also be provided in parallel to allow for a three, four, or more discrete flow resistances. The shunt 120 may be equipped with a valve 1710 that controls flow to the main lumen 1700 and the secondary lumen 1705. The valve position may be controlled by an actuator such as a button or switch on the housing of flow controller 125. The embodiment of FIGS. 13 and 14 has an advantage in that this embodiment in that it maintains precise flow lumen sizes even for the lowest flow setting.

The secondary flow lumen size can be configured to prevent thrombus from forming under even the lowest flow or prolonged flow conditions. In an embodiment, the inner diameter of the secondary lumen 1705 lumen is 0.063 inches or larger.

In an embodiment, the connectors which connect the elements of the reverse flow system are large bore, quick-connect style connectors. For example, a male large-bore hub 680 on the Y-adaptor 660 of arterial sheath 110, as seen in FIG. 9B, connects to a female counterpart 1320 on the arterial side of flow shunt 120. Similarly, a male large bore connector 1310 on the venous side of flow shunt 120 connects to a female counterpart connector 1310 on the flow line of venous sheath 115, as seen in FIG. 10C. The connections can be standard female and male Luer connectors or other style of tubing connectors.

### Sensor(s)

As mentioned, the flow control assembly 125 can include or interact with one or more sensors, which communicate with the system 100 and/or communicate with the patient's anatomy. Each of the sensors can be adapted to respond to a physical stimulus (including, for example, heat, light, sound, pressure, magnetism, motion, etc.) and to transmit a resulting signal for measurement or display or for operating the controller 1130. In an embodiment, the flow sensor 1135 interacts with the shunt 120 to sense an aspect of the flow through the shunt 120, such as flow velocity or volumetric rate of blood flow. The flow sensor 1135 could be directly coupled to a display that directly displays the value of the volumetric flow rate or the flow velocity. Or the flow sensor 1135 could feed data to the controller 1130 for display of the volumetric flow rate or the flow velocity.

The type of flow sensor 1135 can vary. The flow sensor 1135 can be a mechanical device, such as a paddle wheel, flapper valve, rolling ball, or any mechanical component that responds to the flow through the shunt 120. Movement of the mechanical device in response to flow through the shunt 120 can serve as a visual indication of fluid flow and can also be calibrated to a scale as a visual indication of fluid flow rate. The mechanical device can be coupled to an electrical component. For example, a paddle wheel can be positioned in the shunt 120 such that fluid flow causes the paddle wheel to rotate, with greater rate of fluid flow causing a greater speed of rotation of the paddle wheel. The paddle wheel can be coupled magnetically to a Hall-effect sensor to detect the speed of rotation, which is indicative of the fluid flow rate through the shunt 120.

The system 100 is not limited to using a flow sensor 1135 that is positioned in the shunt 120 or a sensor that interacts with the venous return device 115 or the arterial access device 110. For example, an anatomical data sensor 1140 can communicate with or otherwise interact with the patient's anatomy such as the patient's neurological anatomy. In this manner, the anatomical data sensor 1140 can sense a measurable anatomical aspect that is directly or indirectly related to the rate of retrograde flow from the carotid artery. For example, the anatomical data sensor 1140 can measure blood flow conditions in the brain, for example the flow velocity in the middle cerebral artery, and communicate such conditions to a display and/or to the controller 1130 for adjustment of the retrograde flow rate based on predetermined criteria. In an embodiment, the anatomical data sensor 1140 comprises a transcranial Doppler ultrasonography (TCD), which is an ultrasound test that uses reflected sound waves to evaluate blood as it flows through the brain. Use of TCD results in a TCD signal that can be communicated to the controller 1130 for controlling the retrograde flow rate to achieve or maintain a desired TCD profile. The anatomical data sensor 1140 can be based on any physiological measurement, including reverse flow rate, blood flow through the middle cerebral artery, TCD signals of embolic particles, or other neuromonitoring signals.

In another safety mechanism, the controller 1130 includes a timer 1170 (FIG. 12) that keeps time with respect to how long the flow rate has been at a high flow rate. The controller 1130 can be programmed to automatically cause the system 100 to revert to a low flow rate after a predetermined time period of high flow rate, for example after 15, 30, or 60 seconds or more of high flow rate. After the controller reverts to the low flow rate, the user can initiate another predetermined period of high flow rate as desired. Moreover, the user can override the controller 1130 to cause the system 100 to move to the low flow rate (or high flow rate) as desired.

### Example Percutaneous Sheath Embodiments

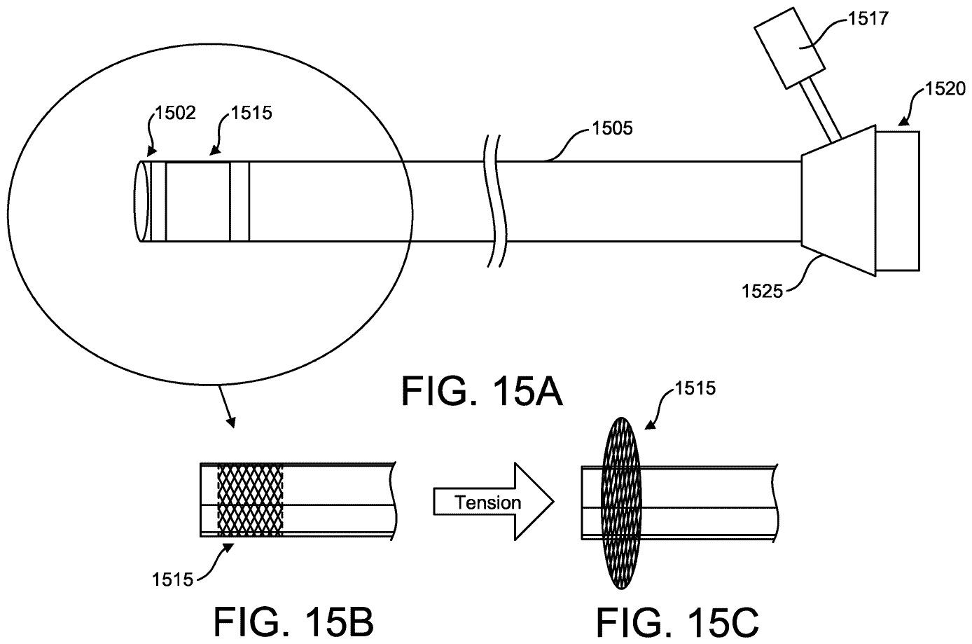

FIG. 15A shows a schematic representation of an embodiment of a sheath 1505 that is configured for percutaneous access and occlusion of a blood vessel such as an artery (for example, the common carotid artery.) The sheath 1505 (or any of the sheaths described herein) may be configured in accordance with or as a supplement to the arterial access device 110 described above. The sheath 1505 has an internal lumen and a distal tip 1502. The sheath 1505 has a distal region 1510 that includes an expandable portion 1515 that is configured to expand radially outward and occlude or partially occlude a blood vessel when positioned in the blood vessel. The expandable portion 1515 includes a plurality of corrugations or other similar structures (such as accordion-like structures or corrugated body that expands and contracts along its length) that can transition between a contracted state and an expanded state wherein the expanded state is suitable for occlusion (or partial occlusion) of a blood vessel. In an embodiment, the expandable portion 1515 is a braid or mesh (such as Nitinol) with a coating such as a polymeric coating or a fabric (such as a polymeric coated fabric) positioned on or over the braid or mesh. In any of the embodiments described herein, the sheath may have angled shape, such as one or more bends or curves, to facilitate centering of the distal tip in the vessel. Such bend(s) may be sharp or acute relative to a long axis of the sheath or the bends may have a soft curved. Such bend(s) may be positioned at a distal tip distal to an expandable element such as a balloon, within the balloon, or immediately proximal to the balloon.

The expandable portion 1515 can be connected to an elongated actuator element, such as one or more tension wires, that run through small or appropriately sized lumens in the wall of the sheath 1505 to a proximal end of the sheath 1505. The actuator element(s) are connected to a control element such as a tension ring 1525 on a proximal hub 1525 of the sheath 1505. The proximal hub 1525 can include a flush port 1517.

The expandable portion 1515 can be transitioned between the contracted state (FIG. 15B) and the expanded state (FIG. 15C) by applying or removing tension (or compression) on the expandable portion 1515 via the actuator element 1520 and the tension ring 1525. As force (tension or compression) is applied, the expandable portion 1515 expands radially into an enlarged disc shape that occludes the blood vessel when positioned in a blood vessel. As the force is released, the expandable portion 1515 relaxes and flattens back out allowing the sheath to be removed from the vessel. Depending on the mechanism, an increase or relief of the applied force may be used to occlude the vessel or remove the occlusion.

A force (tension or compression) may be exerted onto the expandable portion 1515 by actuating the tension ring 1525 such as via a screw or pulley system. It should be appreciated that other mechanisms of creating and reducing tension may also be used, such as for example stretch and lock system, advancing/retracting the braid/mesh into/out of the polymeric material, or other means known to the art.

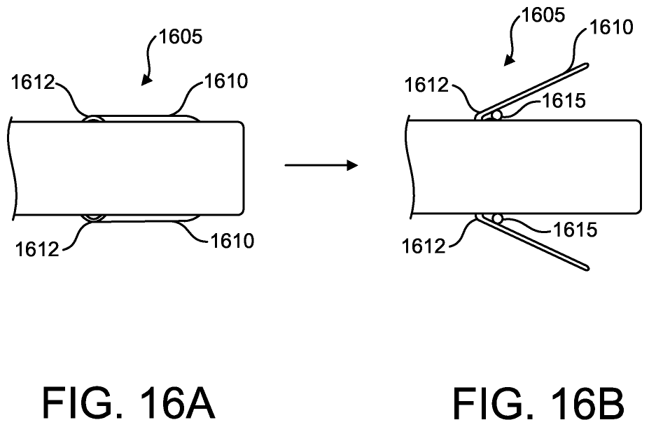

FIG. 16A shows a schematic representation of a distal region of an embodiment of a sheath 1605 that is configured for percutaneous access and occlusion of a blood vessel. This embodiment includes one or more wings or flaps 1610 that are positioned on the distal region of the sheath 1605. The flaps 1610 are attached at a base region 1612 to an outer region of the sheath 1605. One or more expandable members 1615 (such as balloons) are positioned at or near the base region 1612 between an inner surface of a flap and an outer surface of the sheath. In a contracted state, the flaps 1610 are positioned flush or substantially flush with an outer surface of the sheath 1605 such that the flaps 1610 do not contribute or minimally contribute to a diameter of the sheath 1605.

The flaps 1610 can transition to an expanded state (FIG. 16B) wherein the flaps 1610 cantilever outward from the sheath 1605. This results from the expandable members 1615 transitioning to a larger size and pushing the flaps 1610 to the expanded state. The expandable members 1615 can transition to the larger size such as by being inflated via one or more inflation lumens in the sheath 1605. To transition back to the contracted state, the expandable members 1615 are deflated so that they reduce in size. The flaps cinch down to permit the sheath to be pulled out of the vessel. The flaps 1610 may be used in combination with any of the expandable elements (such as expandable balloons) described herein.

The flaps may be made of any of a variety of materials, such a polymeric mateiral, polymer-coated fabric, or other non-porous material that can prevent blood from passing and occlude the vessel. The flaps may overlap with one another around a circumference of the sheath to enable full circumferential occlusion of the vessel.

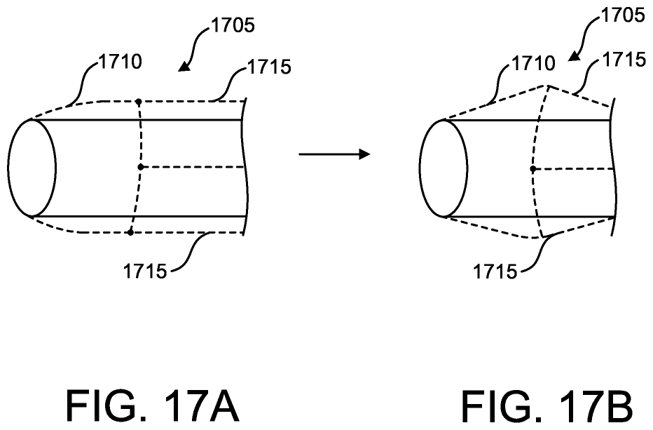

FIG. 17A shows a schematic representation of a distal region of an embodiment of a sheath 1705 that is configured for percutaneous access and occlusion of a blood vessel. This embodiment includes an expandable umbrella element 1710 (shown in phantom) on the distal region wherein the umbrella element extends around an entire circumference of the sheath in an annular fashion. The umbrella element 1710 transitions between a contracted state shown in FIG. 17A and an expanded state shown in FIG. 17B. The umbrella may be made of various materials such as Nitinol or stainless steel (frame), or polymer, polymeric coated fabric, or other non-porous materials (covering). The umbrella element 1710 includes or is attached to one or more tethers 1715 (such as wires, rods, etc.) that extend from the umbrella element 1710 to a proximal hub of the sheath 1705, where a user can actuate the tether(s) 1715 to control expansion and contraction of the umbrella element 1710. The tether 1715 is actuated via pushing, twisting, screwing, or other mechanism at the proximal hub to cause the umbrella element to move between the expanded and contracted state. As the wires advance in a distal direction, they exert a force that causes the umbrella element to opens (i.e., expand) and to occlude the vessel. The tether may have sufficient column strength to permit a user to push the tether such that it exerts a force onto the umbrella element.

In the embodiment of FIGS. 17A and 17B, the umbrella element is located on an outer surface of the sheath 1705. In the retracted, collapsed or non-expanded state shown in FIG. 17A, the umbrella element is positioned flat or substantially flush with an outer surface of the sheath such that the umbrella element does not substantially increase an outer size of the sheath. The tether(s) 1715 can be advanced distally (to the left relative to FIG. 17A and FIG. 17B) to exert a force onto the umbrella element. As the tethers 1715 are advanced, a distal-most tip of the umbrella element remains fixed to the sheath while a proximal region of the umbrella element moves in a distal direction. This causes the umbrella element 1710 to opens out radially into a conical shape, a bi-cone shape or other expanded shape. The tethers can be retracted back to close the umbrella element.

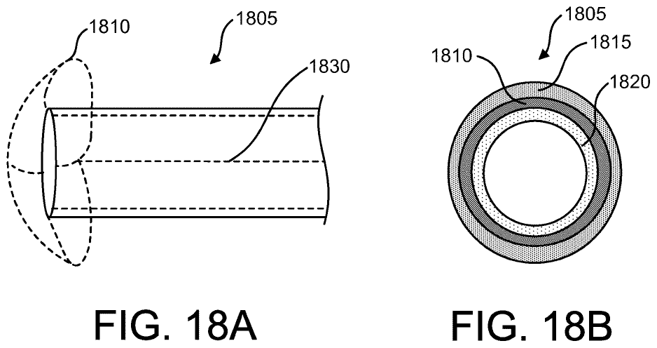

FIG. 18A shows another embodiment of an umbrella element 1810 on a sheath 1805. In this embodiment, the umbrella element 1810 is positioned between an outer sheath layer 1815 and a coaxial inner sheath layer 1820, as shown in the cross-sectional view of FIG. 18B. The umbrella element 1810 is attached to one or more tethers 1830 that can be advanced to cause the umbrella to slide distally outward from between the outer sheath layer 1815 and the coaxial inner sheath layer 1820. The umbrella element expands outward to an expanded state. The umbrella element 1810 can be made of shape-memory wire such as nitinol that expands outward radially as the umbrella element un-sheaths. The tethers 1830 can be retracted to slide or sheath the umbrellas back between the outer sheath layer 1815 and the coaxial inner sheath layer 1820 and thereby retract.

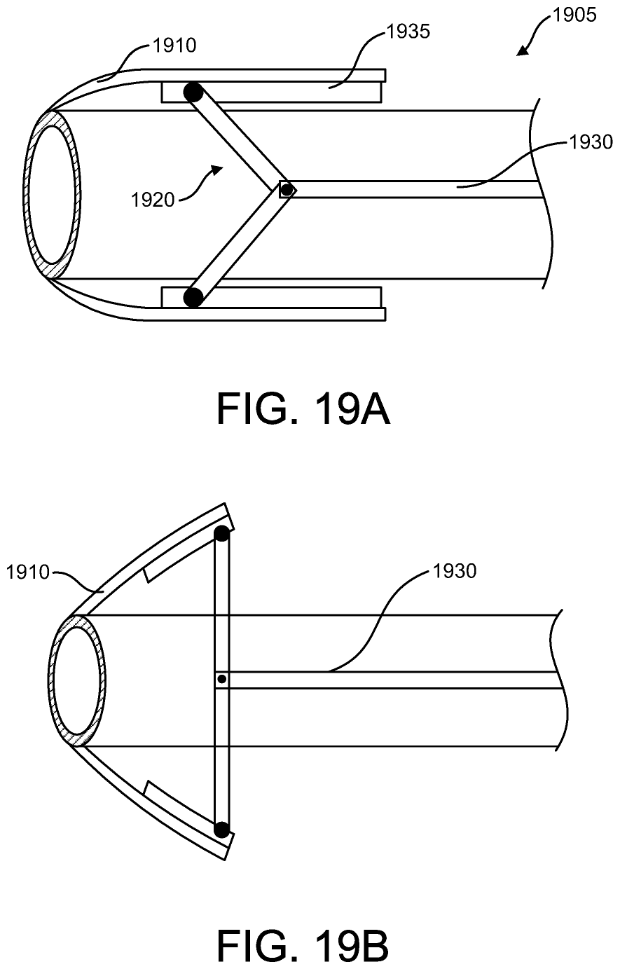

FIGS. 19A and 19B show another embodiment of an umbrella element 1910 on a sheath 1905. In this embodiment, the umbrella element is formed of a frame 1920 coupled to push wires (or similar, e.g. rods) 1930 that extend toward a proximal hub. The metal frame 1920 and push wires 1930 can be slidably positioned between inner and outer sheath layers.

The umbrella element 1910 can be made of a deformable material such as fabric or polymer and is positioned on the outside of the sheath 1905. The umbrella element 1910 includes a slotted track 1935. As the frame 1920 is pushed distally (via the push wires 1930), arms of the frame 1920 are pushed out and slide along the slotted track 1935. This causes the umbrella element to expand as shown in FIG. 19B.

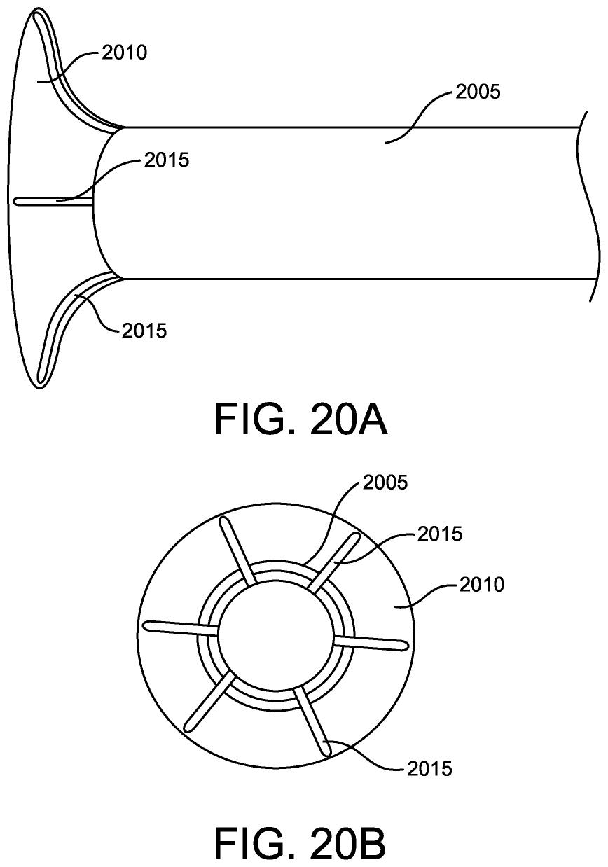

FIGS. 20A and 20B show a distal region of a sheath 2005. An expandable element such as a balloon 2010 is coupled to the distal region of the sheath 2005. The balloon 2010 is shown in an expanded state in FIGS. 20A and 20B such that the balloon 2010 flares outward from an outer wall or a distal edge of the sheath 2005 in a manner that permits the balloon 2010 to at least partially occlude a blood vessel. The sheath 2005 can be formed such that inner and outer walls form an annular cavity that houses the balloon 2010 in a constrained or non-expanded state. Or the balloon 2010 can be constrained within an inner lumen of the sheath 2005 when constrained. In this manner, the sheath 2005 can provide protection to the balloon 2010 such as during insertion of the sheath 2005 into a blood vessel.

The balloon 2010 can vary in configuration. For example, in an embodiment, the balloon 2010 is formed solely of a compliant or malleable material that is configured to expand into a desired shape upon inflation (such as via inflation lumen(s) in the sheath 2005.

In another embodiment, the balloon includes or is coupled to one or more actuation elements 2015 that can be actuated to cause the balloon to transition to the expanded shape. The actuation elements can be for example wires that are attached to or molded within the balloon. The wires can be used to push the balloon 2010 out of the distal region of the sheath 2005 where the balloon can be inflated into the expanded state. The wire(s) may include, for example, a shape memory material (such as Nitinol) that provides scaffolding for the desired shape of the balloon 2010 in the expanded state. The balloon can be deflated and pulled back into the sheath using the wires to transition the balloon 2010 to the constrained state such as during withdrawal of the sheath 2005 from the blood vessel.

In another embodiment, the balloon 2010 is molded around one or more shape memory wires that flare the balloon 2010 outward to the vessel wall as the balloon expands. As the balloon is deflated, a vacuum pressure guides the wires back into the sheath.

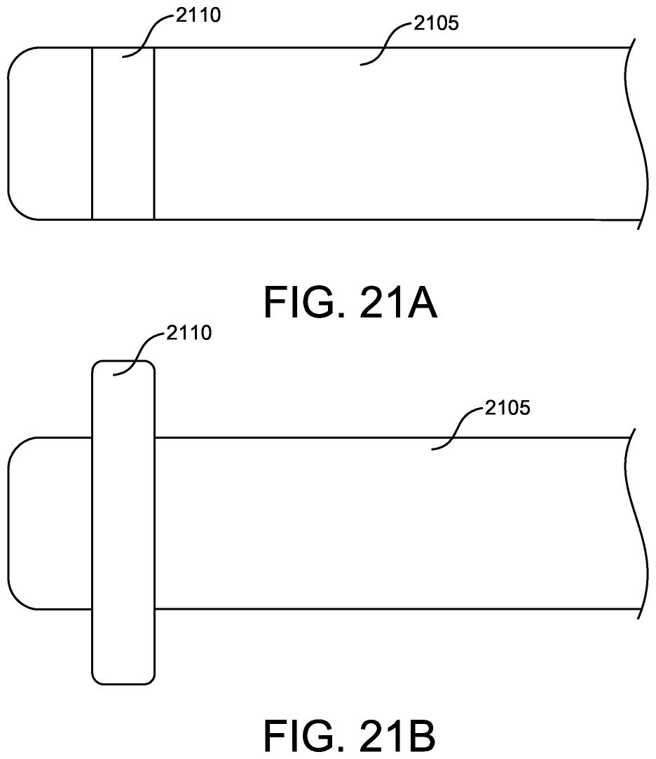

FIGS. 21A and 21B show another embodiment of a distal region of a sheath 2105 that includes an expandable balloon 2110 formed of a compliant ring that is integrated into an outer wall of the sheath 2005. The balloon 2110 is a deformable, annular structure that extends circumferentially around an outer wall of the sheath. The sheath 2005 includes one or more inflation lumen(s) that can be used to expand the balloon from a constrained or smaller sized shape (shown in FIG. 21A) to an expanded shape (shown in FIG. 21B). The inflation lumen may be co-axial with the sheath or non-co-axial. In a method of manufacture, the balloon is attached to the sheath 2005 using a laser bonding process. A laser is used to heat the material of the balloon to cause the balloon to fuse or bond with the distal region of the sheath. The balloon may be made or a flexible, compliant material and the sheath is made of a polymer in an embodiment.

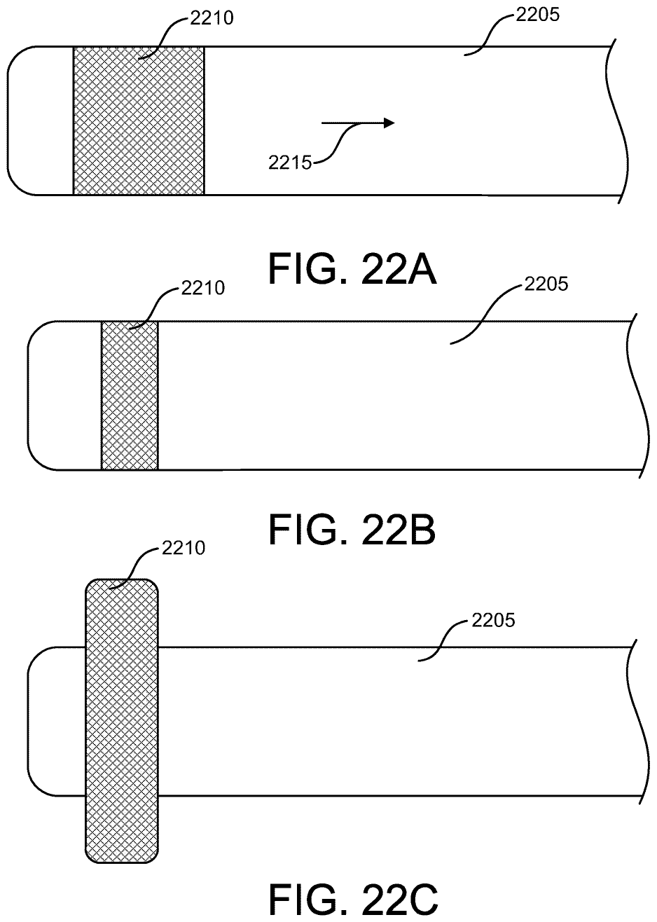

FIGS. 22A-22C show another embodiment of a distal region of a sheath 2205 that includes an expandable balloon 2210, which is integrated into an outer wall of the sheath 2205. For example, the balloon 2210 may sit on the outer wall or it may be at least partially positioned within the outer wall such as between an inner layer and an outer layer of the outer wall of the sheath. The expandable balloon 2210 is coupled with the outer wall such that the balloon 2210 does not increase a diameter of the sheath when the balloon 2210 is in a contracted or unexpanded state as shown in FIG. 22A. The balloon 2210 is made of a compliant material that can be stretched along a long axis of the sheath 2205 as shown in FIG. 22A, which shows the balloon 2210 in the stretched state (stretched along the length of the sheath.) This stretched configuration eliminates or reduces folds or undulations in the balloon. The stretched configuration also places the balloon in tension 2210 such that the outer diameter of the balloon 2210 (and the sheath 2205) is reduced or minimized such as during insertion of the sheath 2205 into the blood vessel.

With reference to FIG. 22B, the tension in the balloon 2210 can be relaxed or otherwise releases such that the length of the balloon 2210 along a longitudinal axis of the sheath 2205 decreases relative to the stretched configuration shown in FIG. 22A. With the balloon 2210 no longer in tension, the material of the balloon 2210 now has the ability to expand radially outward. The balloon 2210 can then be inflated (such as via one or more inflation lumens in the sheath 2205) to cause the balloon 2210 to transition to an expanded state of increased outer diameter as shown in FIG. 22C.

The stretched configuration of the balloon 2210 shown in FIG. 22A can be achieved in various manners. For example, by pulling proximally back (as exhibited by arrow 2215) on an outer portion of the sheath 2205 so that it pulls and stretches an attached portion of the balloon 2210. The outer portion of the sheath 2205 can then be locked into position with a lock element such as a latch, screw, lock, hook etc. located at a proximal hub of the sheath 2205.

There is now described non-limiting examples of mechanisms that can be coupled to the sheath 2205 for controlling the tension configuration of the balloon 2210.

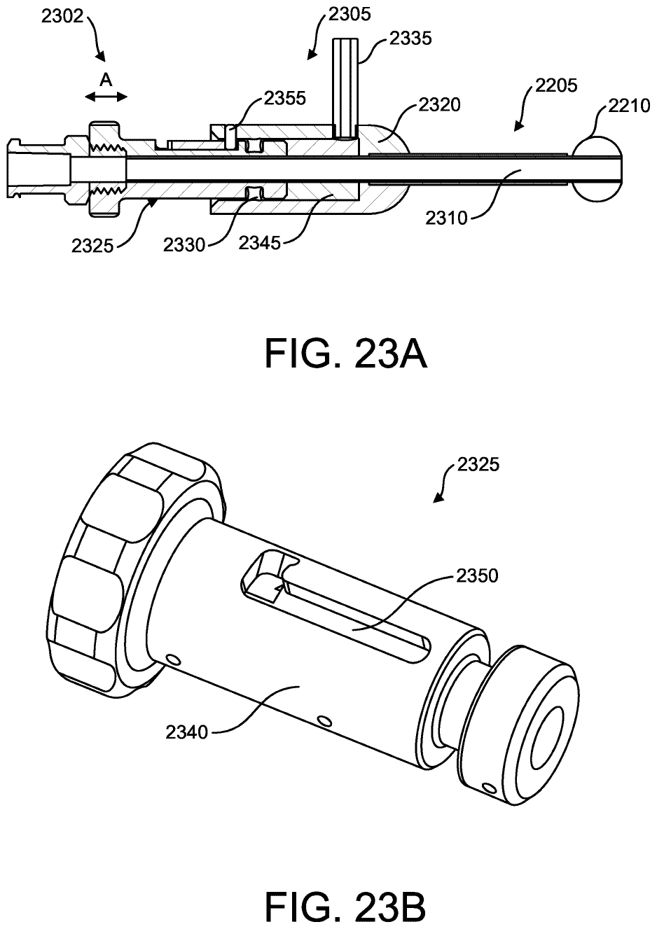

FIG. 23A shows a first mechanism 2305 that is incorporated or otherwise coupled with a proximal hub 2302 of the sheath 2205, which includes an inner shaft 2310 and a co-axial outer shaft 2315 that is slidable relative to the inner shaft 2310. The hub 2302 includes a first component 2320 attached to the outer shaft 2315 and a second component 2325 attached to the inner shaft 2310. A seal element 2330, such as an o-ring, provides a sealed relationship between the first component 2320 and the second component 2325. The balloon is attached at a proximal end to the outer shaft 2315 and at a distal end to the inner shaft 2310 such that relative movement between the inner shaft 2310 and the outer shaft 2315 can be used to place the balloon 2210 in the stretched configuration. The hub 2302 also includes an inflation pathway 2335 that can be used to inflate the balloon 2210.

FIG. 23B shows a perspective view of the second component 2325. The second component 2325 has an elongate body 2340 that is slidably positioned within a cavity 2345 (FIG. 23A) in the first component 2320 such that the second component 2325 can slide along a long axis of the sheath 2205 relative to the first component 2320 as represented by the arrow A in FIG. 23A. With reference to FIG. 23B, the body 2340 of the second component 2325 has translation pathway 2350 in which a pin 2355 of the first component 2320 is positioned. The translation pathway 2350 and pin 2355 are engaged with one another such that they collectively define the slidable movement between the first component 2320 and the second component 2325.

Prior to inflation of the balloon 2210, the hub 2302 is actuated by translocating the first component 2320 relative to the second component 2325. This causes relative movement between the inner shaft 2310 (which is attached to the balloon 2210) and the outer shaft 2315 (which is also attached to the balloon 2210) so that the user can selectively place the balloon 2210 in a tensioned configuration (as shown in FIG. 22A) or the non-tensioned configuration (as shown in FIG. 22B). The balloon 2210 can be inflated via the inflation pathway 2335 when in the non-tensioned configuration.

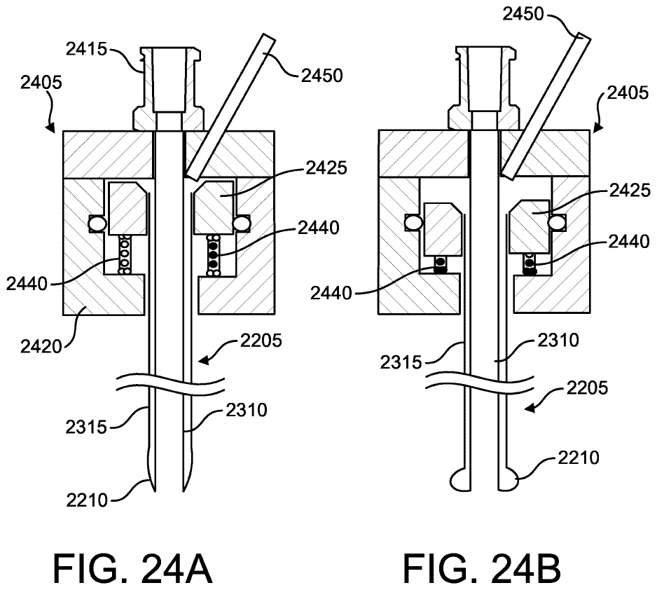

FIGS. 24A and 24B (which are not to scale) show an alternate mechanism that can be coupled to the sheath 2205 for controlling the tension configuration of the balloon 2210. FIG. 24A shows the balloon 2210 in a contracted state and FIG. 24B shows the balloon 2210 in an expanded state. A hub 2405 is located at a proximal region of the sheath 2205 and includes an access device, such as a Luer element 2415 that provides access to an internal lumen of the sheath 2205. The hub 2405 includes a first hub portion 2420 attached to the inner shaft 2310 and a second hub portion 2425 attached to outer shaft 2315 of the sheath 2205. The inner shaft 2310 is attached to a distal end of the balloon 2210. The outer shaft 2315 is attached to a proximal end of the balloon 2210.

The second hub portion 2425 is slidably positioned within an internal cavity of the first hub portion 2420. The second hub portion 2425 can move within the cavity along a long axis of the sheath 2205. The second hub portion 2425 divides the internal cavity into a proximal portion 2435 and a distal portion 2440. A biasing element such as a spring 2440 is positioned in the second hub portion 2440 to bias the second hub portion 2425 toward the proximal portion 2435 of the internal cavity. A seal can be positioned to provide a sealed separation between the proximal portion 2435 and the distal portion 2440. In addition, an inflation shaft 2450 communicates with the proximal portion 2435 and with the balloon 2210.

The spring 2440 holds a constant tension on the outer shaft 2315 via the second hub portion 2425 such that the balloon 2210 is maintained in a default, contracted state as shown in FIG. 24A. In this state, the balloon 2210 is stretched along its length. The balloon 2210 can be inflated via the inflation shaft 2450, which causes the balloon 2210 to expand outward and thereby proximally retract the outer shaft 2315 as shown in FIG. 24B. Inflation of the balloon via the inflation shaft 2450 also increases the pressure of cavity 2435 which acts like a piston on the second hub portion 2425. This causes the second hub portion 2425 to slide down as allowed by the spring 2440. The pneumatic force on the second hub portion 2425 is then appropriately balanced (and greater than) the resistive spring force of the spring 2440 in order to actuate. The inflation shaft 2450 can also be used to inject fluids such as water/saline/contrast to the inflation lumen (between the outer shaft and inner shaft) and the balloon 2210.

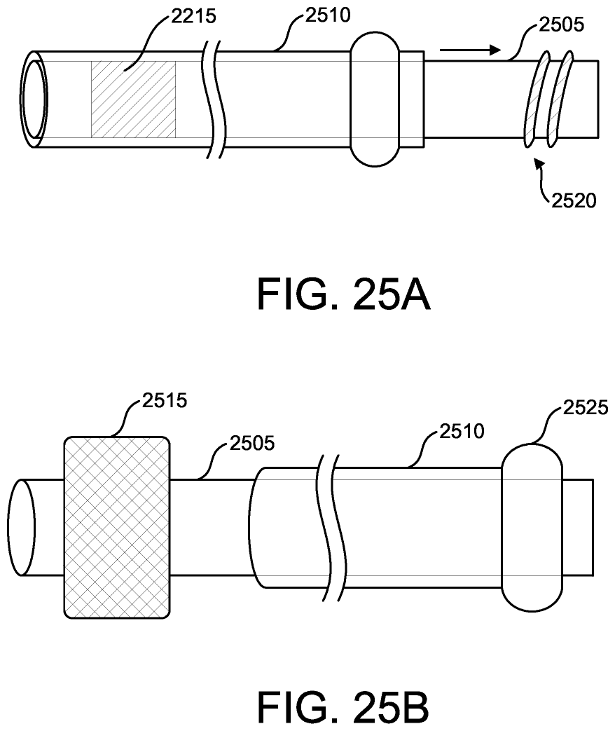

FIGS. 25A and 25B show an embodiment of a sheath system that includes an inner balloon sheath 2505 (or balloon catheter 2505) that is integrated with an outer guide sheath 2510. The guide sheath 2510 is co-axially and slidably positioned over the balloon sheath 2505 such that the balloon sheath is slidably and removably positioned inside an internal lumen of the guide sheath. The balloon sheath 2505 includes an expandable element, such as a balloon 2515, that can transition between a contracted state and an expanded state. The balloon 2515 extends around a circumference of the outer wall of the sheath. FIG. 25A shows the system in a first state or position wherein the guide sheath 2510 is positioned relative to the balloon sheath 2505 such that a portion of the guide sheath 2510 covers and constrains the balloon 2510 in the contracted state. That is, the guide sheath 2510 covers the balloon 2215 such that an inner wall of the guide sheath 2510 constrains the balloon 2215 from outward expansion. The inner catheter 2505 comprises a lumen extending between a proximal end and a distal end, the distal end adapted to receive blood flow from a common carotid artery. The outer guide sheath and the inner catheter 2505 are adapted to be collectively introduced through a puncture in the common carotid artery or another artery such as the femoral artery. Any of the embodiments described herein can be introduced through the common carotid artery, the femoral artery, or other artery.

FIG. 25B shows the system in a second state or position wherein the guide sheath 2510 has been axially moved (along a long axis of the sheath such as in a proximal direction or rightward relative to FIG. 25B) relative to the balloon sheath 2505 (or vice-versa) such that the guide sheath 2510 no longer covers or constrains the balloon 2515. The relative movement between the guide sheath and balloon sheath be via movement of only the guide sheath, only the balloon sheath, or both the guide sheath and balloon sheath. A distal edge of the guide sheath 2510 is thus positioned proximal of a proximal edge of the balloon 2515 in FIG. 25B. The balloon 2515 is free to expand outward such as via inflation, via a self-bias toward expansion, or other mechanism. The balloon sheath 2505 can include a locking element such as a thread arrangement 2520 that mechanically interfaces with a corresponding locking element 2525 (such as corresponding threads) on the guide sheath 2510. That is, the outer wall of the balloon sheath 2505 has one or more threads that interact with complementary threads on an inner wall of the locking element 2525 of the outer guide sheath 2510. In an embodiment, the locking element 2525 is an annular collar that has an expanded outward size relative to at least a portion of the outer guide sheath 2510 such as an adjacent region of the outer guide sheath 2510 on which the collar is positioned. The collar can rotate about an outer wall and/or long axis of the outer guide sheath 2510 relative to the remainder of the outer guide sheath 2510. This permits a user to rotate the locking element 2525 and lockingly engage the threads of the guide sheath with the threads of the inner balloon catheter 2505. Thus, the locking collar has a first set of threads on an inner wall of the locking collar, wherein the first set of threads engage a second set of threads on an outer wall of the inner catheter and lock a position of the inner catheter relative to the outer guide sheath. In an embodiment, the locking collar aligns with the second set of threads on the guide sheath when the inner catheter is in the first position. In another embodiment, there is a third set of threads (or a continuous thread configuration along the inner catheter) on the guide sheath wherein the locking collar aligns with threads when in the second position.

FIG. 25B shows the guide sheath 2510 with the locking element 2525 of the guide sheath positioned to lock with the threads 2520 (FIG. 25A) of the balloon sheath. When in the configuration shown in FIG. 25A, the guide sheath 2510 can also protect the balloon 2515 such as during transit through tissue. As mentioned, the collar 2525 can be rotated to cause the threads to engage one another and lock the relative positions of the guide sheath and balloon catheter to one another.

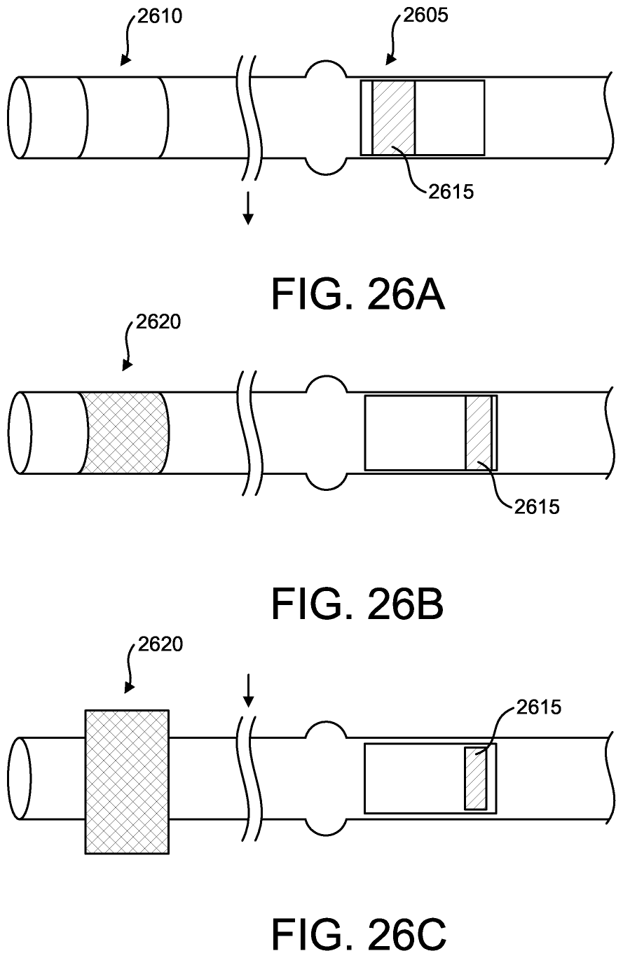

FIGS. 26A-26C show another embodiment of a sheath system that includes a balloon sheath 2605 (or catheter) that is integrated with a guide sheath. With reference to FIG. 26A, the sheath 2605 includes a window cover 2610 that is aligned with an internal balloon. As described in more detail below, the window cover 2610 can be a layer of material positioned in between an outer housing of the sheath 2605 and an inner layer of the sheath.

FIG. 26A shows the window cover 2610 in a closed state such that the window cover 2610 covers and constrains the internal balloon. The window cover 2610 includes a proximal region 2615 that can be actuated such as by a user retracting the proximal region 2615. This also proximally retracts the window cover 2610 to expose an opening in which the balloon 2620 is positioned. The balloon 2620 can then be inflated to an expanded state as shown in FIG. 26C. Various mechanisms can be used to retract the window cover 2610 such as a slidable pull tab, a trigger, a screw mechanism, a button, etc.

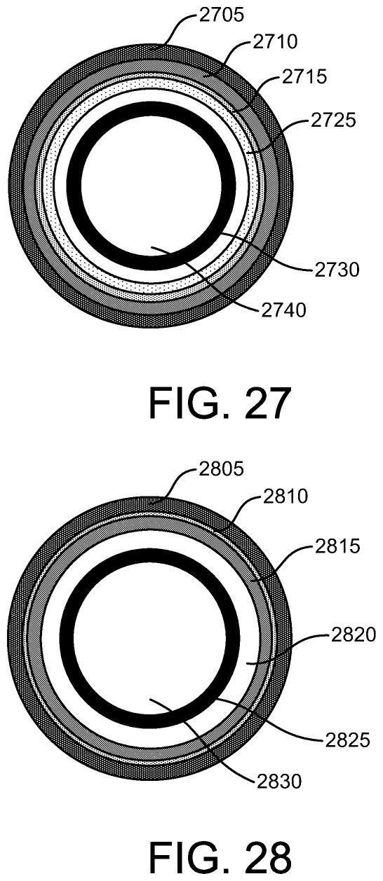

FIG. 27 shows a schematic, cross-sectional view of an embodiment of the sheath 2605 of FIGS. 26A-26C. The sheath 2605 includes an outer layer that forms outer housing 2705. A window layer 2710 (such as a polymer) is positioned inside the outer housing and forms the window cover 2610. A balloon layer 2715 is positioned inside the window layer 2710 and forms the balloon 2620. A middle layer 2720 is formed of a polymer and is positioned beneath the balloon layer 2715. An inflation lumen 2725 is formed between the middle layer 2720 and an inner layer 2730, which may be for example by a braid and/or coil. An inner lumen 2740 is inside the sheath 2605.

FIG. 28 shows a schematic, cross-sectional view of another embodiment of the sheath 2605 of FIGS. 26A-26C. This embodiment of the sheath 2605 includes an outer layer that forms outer housing 2805. A balloon layer 2820 (such as a polymer) is positioned between the outer housing 2805 and a window layer 2815. An inflation lumen is positioned between the window layer 2815 and an inner layer 2825, which can be formed for example by a braid and/or coil. An inner lumen 2830 is inside the sheath 2605.

A window layer 2710 (such as a polymer) is positioned inside the outer housing and forms the window cover 2610. A balloon layer 2715 is positioned inside the window layer 2710 and forms the balloon 2620. A middle layer 2720 is formed of a polymer and is positioned beneath the balloon layer 2715. An inflation lumen 2725 is formed between the middle layer 2720 and an inner layer 2730, which may be for example a braid and/or coil. An inner lumen 2740 is inside the sheath 2605.

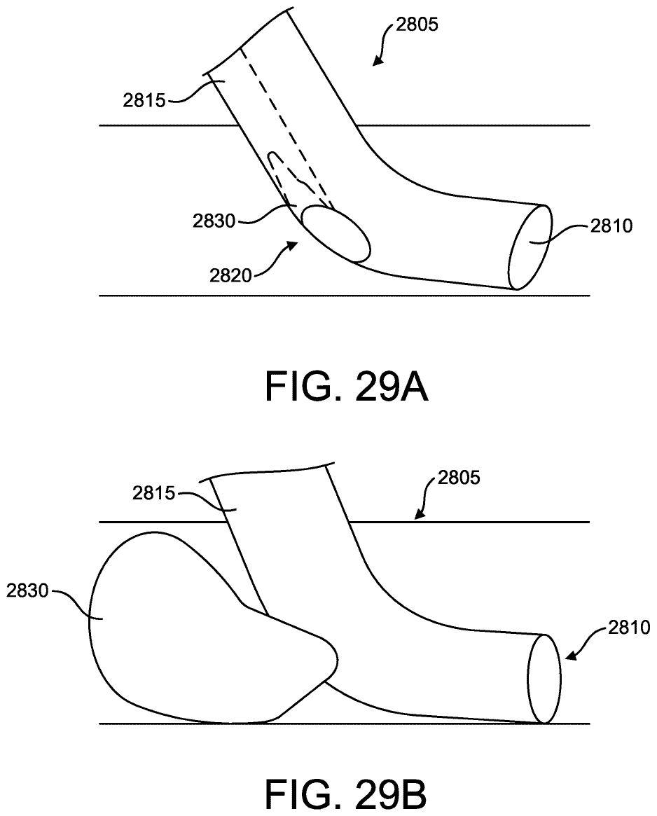

FIGS. 29A and 29B show another embodiment of a sheath 2805 (or inner balloon catheter) positioned in a blood vessel. The sheath 2805 has a bent distal region wherein the bend can be a fixed bend that stays in place unless acted upon to remove the bend. The bend can be a default state of the sheath or the bend can be achieved after placement in the blood vessel. With reference to FIG. 29A, the sheath 2805 includes a primary lumen 2810 that opens at a distal end of the sheath 2805. The sheath also includes a secondary lumen 2815 that is side-by-side with the primary lumen 2810. The secondary lumen 2815 forms a hole or an opening 2820, which is positioned at a distance away from the distal end of the sheath 2805. In an example embodiment, the opening 2820 is 1-5 cm away from the distal end of the sheath 2805 such as at a location of the bend, or 1-2cm. 1-3, cm or 2-3 cm An inflatable balloon 2830 is bonded or otherwise fixed positioned inside the secondary lumen 2815 adjacent the opening 2820. When positioned inside the secondary lumen 2815 as shown in FIG. 29A, the balloon 2830 is in a deflated state of reduced size.

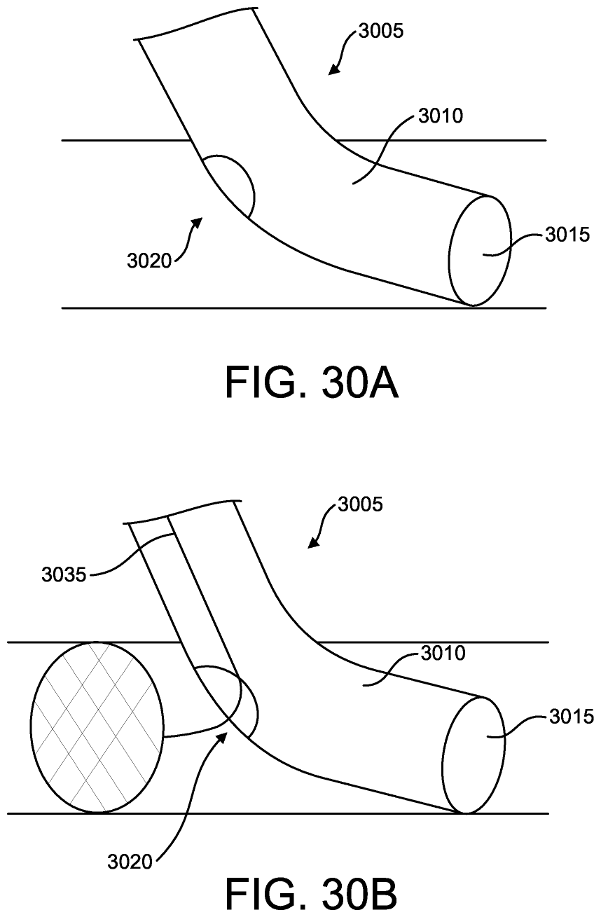

The balloon 2830 can also transition to be positioned outside the secondary lumen 2815 so that the balloon 2830 is positioned flush with an outer surface of the sheath 2805 so that the balloon blocks the opening 2820. The balloon 2820 can further be moved outward of the secondary lumen 2815 and outside the sheath 2805 so that it engages an interior of the blood vessel such as to occlude or at least partially occlude the blood vessel. The balloon 2830 is made of a compliant or semi-compliant material. For example, the balloon 2830 may be made of silicone, polyblend, urethane, Pellethane, or other compliant material. The embodiment of FIGS. 29A-30B can be used in conjunction with the configuration shown in FIGS. 25A and 25B (or any other embodiment) for constraining the balloon 2830.

During insertion of the sheath 2805 into the vessel, the balloon 2830 is structurally protected by either being inside the secondary lumen 2815 or by being taut along the outer surface of the sheath 2805. Once positioned at a desired location in the vessel, the balloon 2830 can be inflated to an expanded state via the secondary lumen 2815 as shown in FIG. 29B. During inflation, the balloon 2830 expands outwardly through the opening 2820. The balloon 2830 can expand to a size that occludes the vessel. The balloon expands in a proximal direction while the primary lumen 2810 opens toward a distal direction. This arrangement may permit additional working space within the vessel. As mentioned, the balloon can be positioned at the bend. In an embodiment, the balloon inflates or extends outward from the sheath in a direction opposite the bend direction as shown in FIG. 29B (or in a direction opposite from the direction that the opening of the lumen 2810 faces)e.

FIGS. 30A and 30B show another embodiment of a sheath 3005 positioned in a blood vessel. This embodiment is similar to the embodiment of FIGS. 29A and 29B although the sheath 3005 may have a single lumen 3010 or dual lumen (not shown). The sheath 3005 has a distal opening 3015 at a distal end of the sheath 3005. The sheath 3005 also has a second opening 3020 that communicates with the lumen 3010 (in the single lumen configuration). The lumen 3010 can be used to deliver one or more therapies via the distal opening 3015, such as for example a stent delivery system, an aspiration catheter, a stent retriever, etc.

The secondary opening 3020 can be used to deliver an occlusion device 3030 which can be mounted on a tether, delivery wire, or delivery shaft 3035, as shown in FIG. 30B. The delivery wire can be used to move the balloon inside and outside of the sheath 3005 via the opening 3020. The occlusion device can be any device that is expandable to occlude the vessel. The occlusion device 3030 can be deployed to occlude the vessel and then the therapy is delivered via the distal opening 3015.

The occlusion device 3030 can vary in configuration. For example, the occlusion device can be a balloon on delivery shaft 3035 formed of a thin wire or tube, which can be hollow for inflation of the occlusion device. The delivery shaft 3035 can have a J-shaped distal region (or other shape) to facilitate passage through the secondary opening 3020. The shape of the occlusion device 3030 can vary. In another example, the occlusion device 3030 is an umbrella-shaped or an accordion-like device coupled to an actuation element such as tension wires for expansion of the occlusion device. A dilator can be used to facilitate delivery of the delivery shaft 3035. Such a dilator can have an inner lumen through which the occlusion device 3030 and delivery shaft 3035 are deployed. The dilator can be used to direct the occlusion system through the secondary opening 3020 to provide automatic entry into the blood and prevent the occlusion system from hitting the blood vessel wall at a sharp angle. The dilator can also contain a bleed back indicator to provide confirmation that the occlusion system was properly inserted into the blood vessel. The occlusion system shown in FIGS. 30A and 30B can also be delivered via the dual-lumen sheath shown in FIGS. 29A and 29B.

### Exemplary Methods of Use

Flow through the carotid artery bifurcation at different stages of the methods of the present disclosure will be described. Initially, the distal sheath 605 (or any embodiment of the percutaneous sheaths described herein) of the arterial access device 110 is introduced into the common carotid artery CCA. As mentioned, entry into the common carotid artery CCA can be via a transcarotid or transfemoral approach, and can be either a direct surgical cut-down or percutaneous access. After the sheath 605 of the arterial access device 110 has been introduced into the common carotid artery CCA, the blood flow will continue in antegrade direction AG with flow from the common carotid artery entering both the internal carotid artery ICA and the external carotid artery ECA.

The venous return device 115 is then inserted into a venous return site, such as the internal jugular vein IJV or femoral vein. The shunt 120 is used to connect the flow lines 615 and 915 of the arterial access device 110 and the venous return device 115, respectively (as shown in FIG. 1A). In this manner, the shunt 120 provides a passageway for retrograde flow from the atrial access device 110 to the venous return device 115. In another embodiment, the shunt 120 connects to an external receptacle 130 rather than to the venous return device 115, as shown in FIG. 1C.

Once all components of the system are in place and connected, flow through the common carotid artery CCA is stopped, such as by using an expandable occlusion element of the percutaneous sheath in the common carotid artery CCA. Alternately, the occlusion element 129 is introduced on second occlusion device 112 separate from the distal sheath 605 of the arterial access device 110, as shown in FIG. 2B. The ECA may also be occluded with a separate occlusion element, either on the same device 110 or on a separate occlusion device.

At that point retrograde flow RG from the external carotid artery ECA and internal carotid artery ICA will begin and will flow through the sheath 605, the flow line 615, the shunt 120, and into the venous return device 115 via the flow line 915. The flow control assembly 125 regulates the retrograde flow as described above. While the retrograde flow is maintained, a stent delivery catheter 2110 (or other intervention device) is introduced into the sheath 605. The stent delivery catheter 2110 is introduced into the sheath 605 through the hemostasis valve 615 and the proximal extension 610. The stent delivery catheter 2110 is advanced into the internal carotid artery ICA and a stent 2115 deployed at the bifurcation B.

Optionally, while flow from the common carotid artery continues and the internal carotid artery remains blocked, measures can be taken to further loosen emboli from the treated region. For example, mechanical elements may be used to clean or remove loose or loosely attached plaque or other potentially embolic debris within the stent, thrombolytic or other fluid delivery catheters may be used to clean the area, or other procedures may be performed. For example, treatment of in-stent restenosis using balloons, atherectomy, or more stents can be performed under retrograde flow. In another example, the occlusion balloon catheter may include flow or aspiration lumens or channels which open proximal to the balloon. Saline, thrombolytics, or other fluids may be infused and/or blood and debris aspirated to or from the treated area without the need for an additional device. While the emboli thus released will flow into the external carotid artery, the external carotid artery is generally less sensitive to emboli release than the internal carotid artery. By prophylactically removing potential emboli which remain, when flow to the internal carotid artery is reestablished, the risk of emboli release is even further reduced. The emboli can also be released under retrograde flow so that the emboli flows through the shunt 120 to the venous system, a filter in the shunt 120, or the receptacle 130.

After the bifurcation has been cleared of emboli, the occlusion element 129 or alternately the tourniquet 2105 can be released, reestablishing antegrade flow, as shown in FIG. 14E. The sheath 605 can then be removed.

A self-closing element may be deployed about the penetration in the wall of the common carotid artery prior to withdrawing the sheath 605 at the end of the procedure.

Usually, the self-closing element will be deployed at or near the beginning of the procedure, but optionally, the self-closing element could be deployed as the sheath is being withdrawn, often being released from a distal end of the sheath onto the wall of the common carotid artery. Use of the self-closing element is advantageous since it affects substantially the rapid closure of the penetration in the common carotid artery as the sheath is being withdrawn.

Such rapid closure can reduce or eliminate unintended blood loss either at the end of the procedure or during accidental dislodgement of the sheath. In addition, such a self-closing element may reduce the risk of arterial wall dissection during access. Further, the self-closing element may be configured to exert a frictional or other retention force on the sheath during the procedure. Such a retention force is advantageous and can reduce the chance of accidentally dislodging the sheath during the procedure. A self-closing element eliminates the need for vascular surgical closure of the artery with suture after sheath removal, reducing the need for a large surgical field and greatly reducing the surgical skill required for the procedure.

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

1. A system for use in accessing and treating a carotid artery, the system comprising:
an outer guide sheath configured to be percutaneously delivered into a carotid artery;
an inner catheter movably positioned inside the outer guide sheath, the inner catheter having an expandable element positioned on a distal region of the inner catheter, wherein the inner catheter comprises a lumen extending between a proximal end and a distal end adapted to receive blood flow from a common carotid artery, the outer guide sheath and inner catheter adapted to be collectively introduced into the common carotid artery, the expandable element adapted to expand and occlude the common carotid artery; and
a locking collar positioned on a proximal region of the outer guide sheath, the locking collar configured to rotate about an outer wall of the outer guide sheath, wherein the locking collar has a first set of threads on an inner wall of the locking collar, and wherein the first set of threads engage a second set of threads on an outer wall of the inner catheter, and wherein the locking collar rotates to cause the first set of threads to engage the second set of threads and lock a position of the inner catheter relative to the outer guide sheath;
wherein the inner catheter is movably positioned inside the outer guide sheath between a first position wherein the outer guide sheath is positioned relative to the outer guide sheath such that a portion of the outer guide sheath covers and constrains the expandable element in a contracted state, and a second position wherein outer guide sheath does not constrains the expandable element, and wherein the locking collar aligns with the second set of threads when the inner catheter is in the first position.

2. The system of claim 1, further comprising a shunt fluidly connected to the inner catheter, wherein the shunt provides a pathway for blood to flow from the inner catheter to a return site.

3. The system of claim 1, wherein the expandable element is a balloon that extends around a circumference of the outer wall of the sheath.

4. The system of claim 1, wherein the inner catheter has a bend at a distal region of the inner catheter.

5. The system of claim 4, wherein the expandable element is a balloon that can be extended out of hole in the inner catheter, wherein the hole is located at the bend.

6. The system of claim 5, wherein the balloon is fixedly attached to the inner catheter near the bend and wherein the balloon can reside entirely within the inner catheter.

7. The system of claim 6, wherein the balloon is fixedly attached to the inner catheter near the bend and wherein the balloon can be positioned flush with an outer wall of the inner catheter and the balloon blocks the hole at the bend.

8. The system of claim 5, wherein the distal end of the inner catheter has an opening and wherein the opening faces a first direction and the balloon extends outward of the hole in a direction opposite the first direction.

9. The system of claim 5, wherein the balloon is positioned inside the inner catheter adjacent the opening.

10. The system of claim 5, wherein the balloon is attached to a delivery wire.

11. The system of claim 10, wherein the delivery wire has an inflation lumen through which the balloon can be inflated.

12. The system of claim 10, wherein the delivery wire can be used to move the balloon inside and outside of the inner catheter via the opening.

13. The system of claim 5, the inner catheter has a primary lumen and a secondary lumen, and wherein the balloon is positioned at least partially in the secondary lumen.

14. The system of claim 5, wherein the hole is located 1-5 cm away from the distal end of the balloon catheter.

15. The system of claim 1, wherein the outer guide sheath and inner catheter are adapted to be collectively introduced through a puncture in the common carotid artery.

16. The system of claim 1, wherein the locking collar has an expanded outward size relative to at least a portion of the outer guide sheath.

17. The system of claim 1, wherein the system is configured to be percutaneously delivered into a carotid artery via an entry location at a femoral artery.

18. The system of claim 1, wherein the system is configured to be percutaneously delivered into a carotid artery via an entry location in a neck.

19. The system of claim 1, wherein the expandable element is corrugated.

20. A system for use in accessing and treating a carotid artery, the system comprising:
an outer guide sheath having a proximal end region;
an inner catheter having a distal end region, the inner catheter being configured to be movably positioned within the outer guide sheath;
an expandable member disposed on the distal end region of the inner catheter; and
a locking collar disposed on the proximal end region of the outer guide sheath, the locking collar configured to releasably secure the outer guide sheath to the inner catheter;
wherein the inner catheter is configured to shift between a first position wherein the expandable member is disposed within the outer guide sheath a second position wherein the expandable member is disposed distally of a distal end of the outer guide sheath.
